(19) United States
(12) Patent Application Publication
(10) Pub. No.: US 2022/0227792 A1
(43) Pub. Date: Jul. 21, 2022

(54) CARBORANE HYDROXAMIC ACID MATRIX METALLOPROTEINASE AGENTS FOR BORON NEUTRON CAPTURE THERAPY

(71) Applicant: LOYOLA UNIVERSITY OF CHICAGO, Chicago, IL (US)

(72) Inventors: Daniel Paul Becker, Glenview, IL (US); Sebastian Flieger, Glenview, IL (US); Isaac Schwarz, Chicago, IL (US)

(21) Appl. No.: 17/616,559

(22) PCT Filed: Jun. 15, 2020
(86) PCT No.: PCT/US20/37698

§ 371 Date: Dec. 3, 2021

Related U.S. Application Data
(60) Provisional application No. 62/861,666, filed on Jun. 14, 2019.

(51) Int. Cl. C07F 5/02; C07D 401/12; C07D 279/12; C07D 213/42; C07D 265/30

(52) U.S. Cl. CPC C07F 5/027; C07D 401/12; C07D 265/30; C07D 213/42; C07D 279/12

(57) ABSTRACT

Disclosed herein are novel carborane hydroxamic acid matrix metalloproteinase (“MMP”) agents bearing borane-containing moieties and methods for their use in treating or preventing a disease, such as cancer and rheumatoid arthritis. In particular, disclosed herein are compounds of Formula (I) and (II) and pharmaceutically acceptable salt thereof: wherein the substituents are as described.

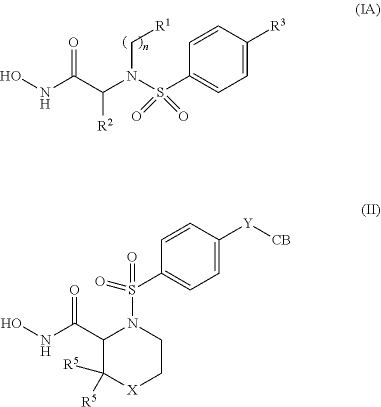

### FIELD

The present disclosure relates to novel carborane hydroxamate matrix metalloproteinase (“MMP”) agents bearing boron-containing moieties that are useful for the treatment of diseases, such as cancer and rheumatoid arthritis.

### BACKGROUND

Matrix metalloproteinases (“MMPs”) are a family of zinc-dependent endopeptidases that are involved in the remodeling and degradation of all components of the extracellular matrix (“ECM”). Birkedal-Hansen et. al., Critical Reviews in Oral Biology and Medicine 4(2):197-250 (1993). MMP enzymes play a key role in normal development, morphogenesis, bone remodeling, wound healing, and angiogenesis. However, inappropriately high MMP activity has been implicated in a number of disease states, such as tumor growth and metastasis, and in the degradation of articular cartilage in arthritis. Martel-Pelletier et. al Best Practice & Research Clinical Rheumatology 15(5):805-829 (2001). In particular, MMPs are known to be overexpressed in tumors and articular cartilage in patients suffering from rheumatoid and osteoarthritis enzymes.

MMP inhibitors have been extensively explored to halt disease progression resulting from exaggerated matrix remodeling mediated by MMPs. Fisher et al., Cancer and Metastasis Reviews, 25(1):115 (2006); Becker et al., Journal of Medicinal Chemistry 53:6653-6680 (2010); Becker et al., J. Med. Chem. 48:6713-6730 (2005). These inhibitors also have been used for the imaging of cancer cells because they can bind tightly to MMP receptors. Freskos et al., Bioorg Med Chem Lett 23:5566-5570 (2013). However, known MMP inhibitors only halt angiogenesis, growth, and metastasis, and must be dosed longer term for inhibitory efficacy. Furthermore, MMP inhibitors still do not directly kill cancer cells, and can lead to the Muscular Skeletal Syndrome (MSS) with longer-term dosing. Fingletonn, Semin Cell Dev Biol. 19(1):61-68 (2008).

Two important and archetypal MMP inhibitors that have advanced into human clinical trials include CGS-23023A (see MacPherson et al., J.Med.Chem. 40:2525 (1997)) and Prinomastat/AG-3340 (see Sorbera et al., Drugs of the Future 25(2):150 (2000); U.S. Pat. Nos. 5,753,653, and 6,153,757), shown below.

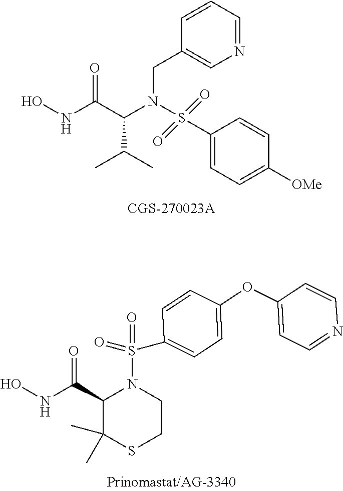 [SMILES: COc1ccc(S(=O)(=O)N(Cc2cccnc2)[C@@H](C(=O)NO)C(C)C)cc1 ; CC1(C)SCCN(S(=O)(=O)c2ccc(Oc3ccncc3)cc2)[C@@H]1C(=O)NO]

However, both of these compounds exhibit musculoskeletal syndrome (“MSS”) side effects in cancer trials (see Zhang et al., Neurother. 8:206 (2011)).

Carboranes are boron cage molecules that have found use in the treatment of diseases, including various cancers and rheumatoid arthritis most notably through boron neutron capture therapy (“BCNT”) and boron neutron capture synovectomy (“BNCS”), respectively. See Valliant et. al., Coord. Chem. Rev 232:173-230 (2002). BNCT is a useful binary cancer treatment, in which a drug containing ¹⁰B atoms is selectively transported into tumor cells and then irradiated with thermal neutrons. A ¹⁰B nucleus adsorbs a neutron to form an excited ¹¹B nucleus, which undergoes decay via fission to emit an α-particle (⁴He²⁺) as well as a ⁷Li³⁺ ion, both with high kinetic energy. These highly charged particles can damage the surrounding tissue. Because these particles have a range of only about one cell diameter (5-9 μm), the radiation damage is limited to the cell in which they arise, thus avoiding damage to the surrounding tissue. Gao et al., Pure Appl.Chem. 87:123-134 (2015). Therefore, BNCT is a potentially promising and precise treatment for cancers.

BNCT is based on the differential absorption of boron in tumor cells—a BNCT agent must concentrate heavily in tumor cells while largely avoiding healthy cells. Current FDA-approved BNCT drugs (e.g., boronophenylalanine, sodium borocaptate and sodium decahydrodecaborate), however, are deficient in that they lack tumor specificit and selectivity, and do not accumulate homogeneously in tumor cells. Ramachandran Future Med. Chem. 5(6):705-714 (2013). Additional BNCT drugs under development are summarized in Barth et al., Radiation Oncology 7:146 (2012). These compounds, like the current FDA-approved BNCT drugs, suffer from a low density of boron atoms (i.e., low neutron-capture cross section), an inability to localize at a tumor site, a lack of bioavailability, poor metabolic stability, or an inability to measure drug concentration in a tumor.

Accordingly, there is a need for new therapeutics having superior specificity and selectivity to treat cancer and chronic inflammation, such as cancer and rheumatoid arthritis caused by abnormally high MMP activity.

### SUMMARY

In one aspect, the disclosure provides a compound having a structure of Formula (I), or a pharmaceutically acceptable salt thereof:

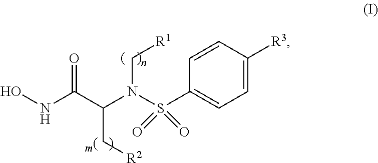

wherein n is 1, 2, or 3; m is 1, 2, or 3; R¹ is either (a) heteroaryl having 5-6 total ring atoms and 1, 2, or 3 heteroatoms selected from N, O, and S, or (b) carboranyl; R² is either (a) C₁₋₆alkyl, (b) carboranyl, or (c) heteroaryl having 5-6 total ring atoms and 1, 2, or 3 heteroatoms selected from N, O, and S, wherein the heteroaryl is substituted with C₀₋₃alkylene-carboranyl; with the proviso that (i) when R¹ is (b), then R² is (a), and (ii) when R² is (b), then R¹ is (a); R³ is H, OH, halo, C₁₋₆alkyl, C₁₋₆alkoxyalkyl, C₁₋₃alkyleneC₆₋₁₀aryl, OC₁₋₆alkyl, OC₁₋₆haloalkyl, OC₁₋₆alkoxyalkyl, OC₀₋₃alkyleneC₆₋₁₀aryl, or N(R⁴)₂; and R⁴ is H or C₁₋₃alkyl. In some cases, n is 1, 2, or 3; m is 1, 2, or 3; R¹ is either (a) heteroaryl having 5-6 total ring atoms and 1, 2, or 3 heteroatoms selected from N, O, and S, or (b) carboranyl; R² is either (a) C₁₋₆alkyl or (b) carboranyl; with the proviso that (i) when R¹ is (b), then R² is (a), and (ii) when R² is (b), then R¹ is (a); R³ is H, OH, halo, C₁₋₆alkyl, C₁₋₆haloalkyl, C₁₋₆alkoxyalkyl, C₁₋₃alkyleneC₆₋₁₀aryl, OC₁₋₆alkyl, OC₁₋₆haloalkyl, OC₁₋₆alkoxyalkyl, OC₀₋₃alkyleneC₆₋₁₀aryl, or N(R⁴)₂; and R⁴ is H or C₁₋₃alkyl.

In another aspect, the disclosure provides a compound having a structure of Formula (IA), or a pharmaceutically acceptable salt thereof:

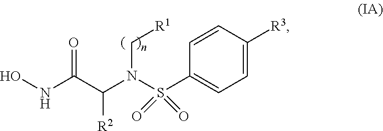

wherein n is 1, 2, or 3; R¹ is either (a) heteroaryl having 5-6 total ring atoms and 1, 2, or 3 heteroatoms selected from N, O, and S, or (b) carboranyl; R² is either (a) C₁₋₆alkyl, (b) C₁₋₃alkylene-carboranyl, or (c) C₁₋₃alkylene-heteroaryl having 5-6 total ring atoms and 1, 2, or 3 heteroatoms selected from N, O, and S, wherein the heteroaryl is substituted with C₁₋₃alkylene-carboranyl; with the proviso that (i) when R¹ is (b), then R² is (a), and (ii) when R² is (b) or (c), then R¹ is (a); R³ is H, OH, halo, C₁₋₆alkyl, C₁₋₆haloalkyl, C₁₋₆alkoxyalkyl, C₁₋₃alkyleneC₆₋₁₀aryl, OC₁₋₆alkyl, OC₁₋₆haloalkyl, OC₁₋₆alkoxyalkyl, OC₀₋₃alkyleneC₆₋₁₀aryl, or N(R⁴)₂; and R⁴ is H or C₁₋₃alkyl.

In some cases, the compound Formula (IA) has a structure,

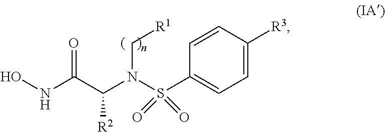

or a pharmaceutically acceptable salt thereof. In various cases, n is 1. In some cases, n is 2. In some embodiments, m is 1. In various embodiments, m is 2. In some embodiments, the carboranyl is ortho-carboranyl. In various embodiments, the carboranyl is meta-carboranyl. In some cases, the carboranyl is para-carboranyl. In some embodiments, the carboranyl is nido-carboranyl. In some cases, R³ is C₁₋₆alkyl, C₁₋₆haloalkyl, OC₁₋₆alkyl, or OC₁₋₆haloalkyl. In various cases, R³ is C₁₋₃alkyl, C₁₋₃fluoroalkyl, OC₁₋₃alkyl, or OC₁₋₃haloalkyl. In some embodiments, R³ is CH₃, CF₃, OCH₃, or OCF₃. In various embodiments, R³ is C₁₋₃alkyleneC₆₋₁₀aryl or OC₀₋₃alkyleneC₆₋₁₀aryl. In some cases, R³ is O-phenyl. In various cases, the phenyl is unsubstituted. In some embodiments, the phenyl is substituted. In some embodiments, n is 1; m is 1; and R³ is OCH₃, OCF₃, or O-phenyl. In various embodiments, R¹ is carboranyl and R² is C₁₋₆alkyl. In some cases, R² is C₁₋₃alkyl. In various cases, R² is isopropyl. In some embodiments, R¹ is heteroaryl having 5-6 total ring atoms and 1, 2, or 3 heteroatoms selected from N, O, and S, and R² is carboranyl. In some cases, R¹ is heteroaryl having 5-6 total ring atoms and 1, 2, or 3 heteroatoms selected from N, O, and S, and R² is C₁₋₃alkylene-carboranyl or C₁₋₃alkylene-heteroaryl having 5-6 total ring atoms and 1, 2, or 3 heteroatoms selected from N, O, and S, wherein the heteroaryl is substituted with C₁₋₃alkylene-carboranyl. In various embodiments, R¹ is pyridinyl. In some cases, wherein R² is C₁₋₃alkylene-carboranyl. In various cases, R² is CH₂-carboranyl. In some embodiments, R² is C₁₋₃alkylene-heteroaryl having 5-6 total ring atoms and 1, 2, or 3 heteroatoms selected from N, O, and S, wherein the heteroaryl is substituted with C₁₋₃alkylene-carboranyl. In various embodiments, heteroaryl is pyridyl, pyrazinyl, pyrimidinyl, pyrrolyl, pyrazolyl, imidazolyl, thiazolyl, triazolyl, oxazolyl, isooxazolyl, thiadiazolyl, oxadiazolyl, furanyl, or thiofuranyl. In some cases, heteroaryl is triazolyl. In various cases, R² is trizolyl substituted with C₃alkylene-carboranyl.

In some embodiments, provided herein are compounds having a structure

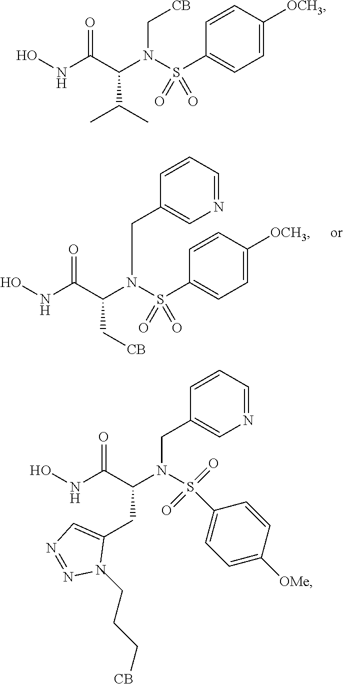

or a pharmaceutically acceptable salt thereof. In some cases, the CB is nido-carboranyl.

In another aspect of the disclosure, provided herein is a compound having a structure of Formula (II), or a pharmaceutically acceptable salt thereof:

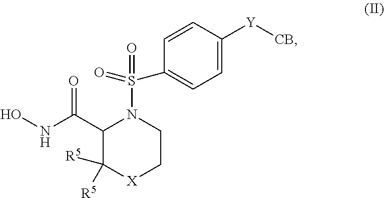

wherein CB is carboranyl; each of X and Y independently is O or S; and each R⁵ independently is H or C₁₋₆alkyl. In some cases, the compound of Formula (II) has a structure:

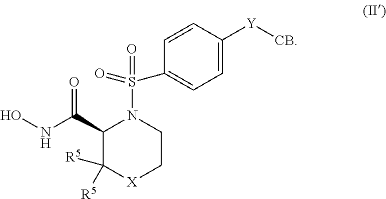

In some embodiments, the carboranyl is ortho-carboranyl. In various embodiments, the carboranyl is meta-carboranyl. In some cases, the carboranyl is para-carboranyl. In some embodiments, the carboranyl is nido-carboranyl. In various embodiments, X is S. In some cases, Y is S. In some embodiments, each R⁵ independently is H. In various cases, each R⁵ independently is C₁₋₆alkyl. In some cases, each R⁵ independently is C₁₋₃alkyl. In various cases, each R⁵ independently is CH₃. In some cases, one R⁵ is H and one R⁵ is CH₃. In various cases, each of X and Y is S and each R⁵ is CH₃. In some embodiments, the compound Formula (II) is

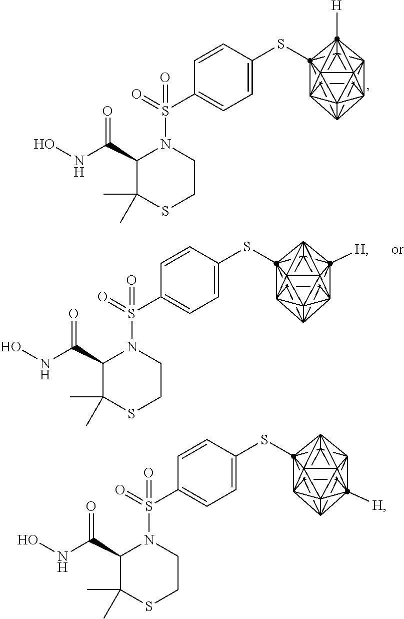

or a pharmaceutically acceptable salt thereof.

Another aspect of the disclosure provides a pharmaceutical formulation comprising the compound or salt described herein and a pharmaceutically acceptable excipient.

Another aspect of the disclosure provides a method of delivering ¹⁰B atoms to matrix metalloproteinase (“MMP”) in a cell, comprising contacting the cell with a compound described herein, wherein the compound binds to MMP with an IC₅₀ of 1 μM or less. In some embodiments, the MMP is MMP-13, MMP-2, MMP-9, or a combination thereof. In various embodiments, the contacting occurs in vivo. In some cases, the contacting comprises administering to a subject in need thereof. In various cases, the subject suffers from cancer, rheumatoid arthritis, or both.

Yet another aspect of the disclosure provides a method of inhibiting matrix metalloproteinase (“MMP”) in a cell, comprising contacting the cell with the compound or salt of described herein, or the composition described herein, in an amount effective to inhibit MMP. In some embodiments, the MMP is MMP-13, MMP-2, MMP-9, or a combination thereof. In various embodiments, the contacting occurs in vivo. In some cases, the contacting comprises administering to a subject in need thereof. In various cases, the subject suffers from cancer, rheumatoid arthritis, or both.

Another aspect of the disclosure provides a method of treating a disease in a subject comprising administering to the subject a therapeutically effective amount of the pharmaceutical formulation described herein. In some embodiments, the disease is cancer or rheumatoid arthritis.

Further aspects of the invention may become apparent to those skilled in the art from a review of the following detailed description, taken in conjunction with the appended claims. While the invention is susceptible of embodiments in various forms, described hereinafter are specific embodiments of the invention with the understanding that the disclosure is illustrative, and is not intended to limit the invention to specific embodiments described herein.

### DETAILED DESCRIPTION

Disclosed herein are orally bioavailable matrix metalloproteinase (“MMP”) agents having a structure of Formula (I) and (II), and pharmaceutically acceptable salts thereof, which bind with high potency and specificity to overexpressed MMP enzymes.

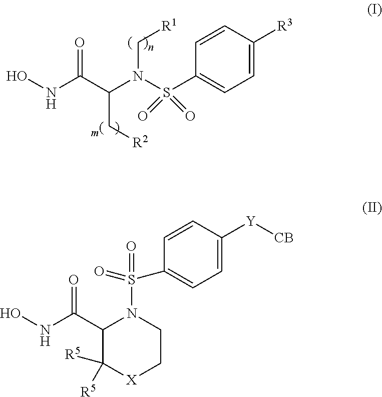

The agents described herein exhibit long half-lives, have good metabolic stability, and low clearance. Because the agents described herein have a high potency for a range of MMP enzymes, such as the collagenase MMP-13 and the gelatinases MMP-2 and MMP-9, they can accumulate in tumors and inhibit angiogenesis, invasion, and metastasis of tumors, as well as MMP-induced destruction of articular cartilage.

The agents described herein have a high neutron-capture cross section, and work by delivering a high density of boron atoms to tumors to enable binary treatment of the tumors using boron neutron capture therapy (“BCNT”). These agents also can deliver a high density of boron atoms to arthritic tissue to treat the tissue using boron neutron capture synovectomy (“BNCS”). The agents described herein are further advantageous because they use the binding potency of the ligands at MMP receptors to target boron atoms into cancer cells, and thus, the exposure to the compounds is only for the duration of the BNCT treatment. In contrast, traditional MMP inhibitors require long-term dosing of the inhibitors to be effective.

### Definitions

As used herein, the term “CB” or “carborane” or “carboranyl” refers to a polyhedron cluster composed of boron, carbon and hydrogen atoms. The carboranyl can be closo-, nido-, arachno-, or hypho-carboranyl. For example, the carboranyl can be a closo-carboranyl or nido-carboranyl. In some cases, the carboranyl is nido-carboranyl. Further, the carbornyl may be an ortho-carboranyl, meta-carboranyl, or para-carboranyl. Any depiction of a compound described herein is exemplary and is intended to include all carboranyl regioisomers. In some embodiments, the carboranyl is ortho. In some embodiments, the carborane is meta. In some cases, CB is unsubstituted. In various cases, CB is substituted. In some embodiments, CB is substituted with a fluoroalkyl group, such as CF₃. As used herein, a carborane can be depicted as

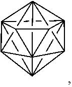

which can represent any regioisomer (e.g., ortho, meta, or para) of any type of carborane (closo-, nido-, arachno-, or hypho-carborane).

As used herein, the term “alkyl” refers to straight chained and branched saturated hydrocarbon groups containing one to thirty carbon atoms, for example, one to twenty carbon atoms, or one to ten carbon atoms. The term Cₙ means the alkyl group has “n” carbon atoms. For example, C₄ alkyl refers to an alkyl group that has 4 carbon atoms. C₁₋₇alkyl refers to an alkyl group having a number of carbon atoms encompassing the entire range (i.e., 1 to 7 carbon atoms), as well as all subgroups (e.g., 1-6, 2-7, 1-5, 3-6, 1, 2, 3, 4, 5, 6, and 7 carbon atoms). Nonlimiting examples of alkyl groups include, methyl, ethyl, n-propyl, isopropyl, n-butyl, sec-butyl (2-methylpropyl), t-butyl (1,1-dimethylethyl), 3,3-dimethylpentyl, and 2-ethylhexyl. Unless otherwise indicated, an alkyl group can be an unsubstituted alkyl group or a substituted alkyl group.

As used herein, the term “haloalkyl” refers to an alkyl group as defined herein that is substituted with one or more halo groups (e.g., F, Cl, Br, I). For example, a fluoroalkyl group is an alkyl group substituted with one or more fluorine atoms. In particular, a C₁₋₆fluoroalkyl group is an alkyl group containing a 1, 2, 3, 4, 5, or 6 carbon atoms with one or more of the carbon atoms substituted with one or more fluorine atoms.

As used herein, the term “alkoxy” refers to an alkyl group, as defined herein, appended to the parent molecule through an oxygen atom. Representative examples of alkoxy include, but are not limited to, methoxy, ethoxy, propoxy, 2-propoxy, butoxy, tert-butoxy, pentyloxy, and hexyloxy.

As used herein, the term “alkoxyalkyl” refers to an alkoxy group, as defined herein, that is appended to the parent molecule through an alkyl group, as defined herein.

As used herein, the term “alkylene” refers to an alkyl group having a substituent. For example, the term “alkylene-aryl” refers to an alkyl group substituted with an aryl group. The term Cₙ means the alkylene group has “n” carbon atoms. For example, C₁₋₆ alkylene refers to an alkylene group having a number of carbon atoms encompassing the entire range, as well as all subgroups, as previously described for “alkyl” groups.

As used herein, the term “aryl” refers to monocyclic or polycyclic (e.g., fused bicyclic and fused tricyclic) carbocyclic aromatic ring systems. Examples of aryl groups include, but are not limited to, phenyl, naphthyl, tetrahydronaphthyl, phenanthrenyl, biphenylenyl, indanyl, indenyl, anthracenyl, and fluorenyl. Unless otherwise indicated, an aryl group can be an unsubstituted aryl group or a substituted aryl group.

As used herein, the term “heteroaryl” refers to monocyclic or polycyclic (e.g., fused bicyclic and fused tricyclic) aromatic ring systems, wherein one to four-ring atoms are selected from oxygen, nitrogen, or sulfur, and the remaining ring atoms are carbon, said ring system being joined to the remainder of the molecule by any of the ring atoms. Nonlimiting examples of heteroaryl groups include, but are not limited to, pyridyl, pyrazinyl, pyrimidinyl, pyrrolyl, pyrazolyl, imidazolyl, thiazolyl, tetrazolyl, oxazolyl, isooxazolyl, thiadiazolyl, oxadiazolyl, furanyl, quinolinyl, isoquinolinyl, benzoxazolyl, benzimidazolyl, and benzothiazolyl. Unless otherwise indicated, a heteroaryl group can be an unsubstituted heteroaryl group or a substituted heteroaryl group.

As used herein, “halo” refers to fluoro, chloro, bromo, or iodo.

As used herein, the term “substituted,” when used to modify a chemical functional group, refers to the replacement of at least one hydrogen radical on the functional group with a substituent. Substituents can include, but are not limited to, alkyl, cycloalkyl, alkenyl, cycloalkenyl, alkynyl, heterocycloalkyl, ether, polyether, thioether, polythioether, aryl, heteroaryl, hydroxyl, oxy, alkoxy, heteroalkoxy, aryloxy, heteroaryloxy, ester, thioester, carboxy, cyano, nitro, amino, amido, acetamide, and halo (e.g., fluoro, chloro, bromo, or iodo). When a chemical functional group includes more than one substituent, the substituents can be bound to the same carbon atom or to two or more different carbon atoms.

As used herein, the term “therapeutically effective amount” means an amount of a compound or combination of therapeutically active compounds (e.g., an MMP agent/inhibitor or combination of MMP agent/inhibitors) that ameliorates, attenuates or eliminates one or more symptoms of a particular disease or condition (e.g., cancer), or prevents or delays the onset of one of more symptoms of a particular disease or condition.

As used herein, the terms “patient” and “subject” may be used interchangeably and mean animals, such as dogs, cats, cows, horses, and sheep (i.e., non-human animals) and humans. Particular patients or subjects are mammals (e.g., humans). The terms patient and subject includes males and females.

As used herein, the term “pharmaceutically acceptable” means that the referenced substance, such as a compound of the present invention, or a formulation containing the compound, or a particular excipient, are safe and suitable for administration to a patient or subject. The term “pharmaceutically acceptable excipient” refers to a medium that does not interfere with the effectiveness of the biological activity of the active ingredient(s) and is not toxic to the host to which it is administered.

As used herein the terms “treating”, “treat” or “treatment” and the like also include preventative (e.g., prophylactic) and palliative treatment.

As used herein, the term “excipient” means any pharmaceutically acceptable additive, carrier, diluent, adjuvant, or other ingredient, other than the active pharmaceutical ingredient (API).

### Carborane Hydroxamic Acid MMP Agents

In one aspect, the agents of the disclosure have a structure of Formula (I), or a pharmaceutically acceptable salt thereof:

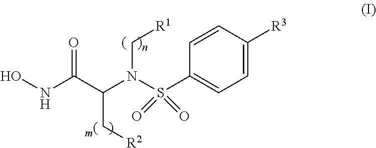

wherein
n is 1, 2, or 3;
m is 1, 2, or 3;
R¹ is either (a) heteroaryl having 5-6 total ring atoms and 1, 2, or 3 heteroatoms selected from N, O, and S, or (b) carboranyl;
R² is either (a) C₁₋₆alkyl, (b) carboranyl, or (c) heteroaryl having 5-6 total ring atoms and 1, 2, or 3 heteroatoms selected from N, O, and S, wherein the heteroaryl is substituted with C₀₋₃alkylene-carboranyl;
with the proviso that
(i) when R¹ is (b), then R² is (a), and
(ii) when R² is (b), then R¹ is (a); and
R³ is H, OH, halo, C₁₋₆alkyl, C₁₋₆haloalkyl, C₁₋₆alkoxyalkyl, C₁₋₃alkyleneC₆₋₁₀aryl, OC₁₋₆alkyl, OC₁₋₆haloalkyl, OC₁₋₆alkoxyalkyl, OC₀₋₃alkyleneC₆₋₁₀aryl, or N(R⁴)₂; and
each R⁴ independently is H or C₁₋₃alkyl. In some cases, n is 1, 2, or 3; m is 1, 2, or 3; R¹ is either (a) heteroaryl having 5-6 total ring atoms and 1, 2, or 3 heteroatoms selected from N, O, and S, or (b) carboranyl; R² is either (a) C₁₋₆alkyl or (b) carboranyl; with the proviso that (i) when R¹ is (b), then R² is (a), and (ii) when R² is (b), then R¹ is (a); R³ is H, OH, halo, C₁₋₆alkyl, C₁₋₆haloalkyl, C₁₋₆alkoxyalkyl, C₁₋₃alkyleneC₆₋₁₀aryl, OC₁₋₆alkyl, OC₁₋₆haloalkyl, OC₁₋₆alkoxyalkyl, OC₀₋₃alkyleneC₆₋₁₀aryl, or N(R⁴)₂; and R⁴ is H or C₁₋₃alkyl.

In another aspect, the agents of the disclosure have a structure of Formula (IA), or a pharmaceutically acceptable salt thereof:

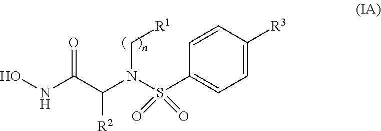

wherein
n is 1, 2, or 3;
R¹ is either (a) heteroaryl having 5-6 total ring atoms and 1, 2, or 3 heteroatoms selected from N, O, and S, or (b) carboranyl;
R² is either (a) C₁₋₆alkyl, (b) C₁₋₃alkylene-carboranyl, or (c) C₁₋₃alkylene-heteroaryl having 5-6 total ring atoms and 1, 2, or 3 heteroatoms selected from N, O, and S, wherein the heteroaryl is substituted with C₁₋₃alkylene-carboranyl;
with the proviso that
(i) when R¹ is (b), then R² is (a), and
(ii) when R² is (b) or (c), then R¹ is (a);
R³ is H, OH, halo, C₁₋₆alkyl, C₁₋₆haloalkyl, C₁₋₆alkoxyalkyl, C₁₋₃alkyleneC₆₋₁₀aryl, OC₁₋₆alkyl, OC₁₋₆haloalkyl, OC₁₋₆alkoxyalkyl, OC₀₋₃alkyleneC₆₋₁₀aryl, or N(R⁴)₂; and
R⁴ is H or C₁₋₃alkyl.

In some embodiments, the compound of Formula (I) has a structure of Formula (I′) and the compound of Formula (IA) has a structure of Formula (IA′):

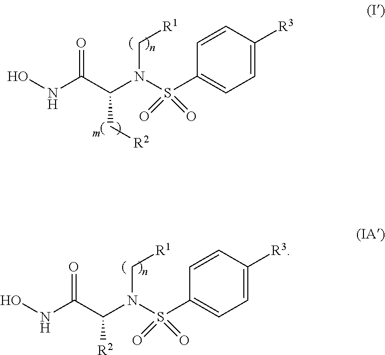

In some cases, n is 1. In various cases, n is 2. In various embodiments, n is 3. In some embodiments, n is 1 or 2.

In some cases, m is 1. In various cases, m is 2. In some embodiments m is 3. In some embodiments, m is 1 or 2.

The carboranyl can be any carboranyl as previously described herein. In some cases, the carboranyl is ortho-carboranyl. In various cases, the carboranyl is meta-carboranyl. In some embodiments, the carboranyl is para-carboranyl. In some cases, the carboranyl is nido-carboranyl.

In some embodiments, R³ is H. In various embodiments, R³ is OH. In some cases, R³ is halo (e.g., F, Cl, Br, or I). For example, R³ can be F or Cl. In some embodiments, R³ is C₁₋₆alkyl. For example, R³ can be methyl, ethyl, propyl, isopropyl, butyl, sec-butyl, isobutyl, tent-butyl, pentyl, 1-methylbutyl, 2-methylbutyl, isopentyl, hexyl, or isohexyl. In some cases R³ is methyl or ethyl. In various cases, R³ is OC₁₋₆alkyl. For example, R³ can be OCH₃, OCH₂CH₃, OCH₂CH₂CH₃, OCH(CH₃)₂, OCH₂CH₂CH₂CH₃, OCH(CH₃)CH₂CH₃, OCH₂CH(CH₃)₂, OC(CH₃)₃, OCH₂CH₂CH₂CH₂CH₃, OCH(CH₃)CH₂CH₂CH₃, OCH₂CH(CH₃)CH₂CH₃, OCH₂CH₂CH(CH₃)CH₃, OCH₂CH₂CH(CH₃)₂, or OCH₂CH₂CH₂CH₂CH₂CH₃. In some embodiments R³ is CH₃ or OCH₃. In various embodiments, R³ is C₁₋₆haloalkyl or OC₁₋₆haloalkyl, such as C₁₋₃haloalkyl or OC₁₋₃haloalkyl. For example, R³ can be CF₃ or OCF₃. In some cases, R³ is C₁₋₆alkoxyalkyl or OC₁₋₆alkoxyalkyl. For example, R³ can be CH₂CH₂OCH₂CH₃ or OCH₂CH₂OCH₂CH₃. In various cases, R³ is N(R⁴)₂. In some embodiments, R⁴ is H or CH₃. For example, R³ can be NH₂, NHCH₃, or N(CH₃)₂. In various embodiments, R³ is C₁₋₃alkyleneC₆₋₁₀aryl or OC₀₋₃alkyleneC₆₋₁₀aryl. Suitable aryl groups include, but are not limited to, phenyl, naphthyl, tetrahydronaphthyl, indanyl, and indenyl. In some cases, aryl is phenyl. In some cases, the phenyl is unsubstituted. In various cases, the phenyl is substituted. In some embodiments, R³ is optionally substituted O-phenyl. Suitable substituents for the aryl group include, for example, halo, C₁₋₆halohalkyl, or OC₁₋₆haloalkyl. For example, the substituent can be Cl, F, CF₃, OCF₃, or OCF₂CF₃.

In some cases for Formula (I), n is 1; m is 1; and R³ is OCH₃, OCF₃, or O-phenyl. In some cases for Formula (IA), n is 1 and R³ is OCH₃, OCF₃, or O-phenyl.

In some embodiments, R¹ is carboranyl, as previously described herein, and R² is C₁₋₆alkyl. In some cases, R² is C₁₋₃alkyl. Suitable R² groups include, for example, methyl, ethyl, propyl, and isopropyl. In some cases, R² is isopropyl. In some embodiments, the compound of Formula (I) has a structure:

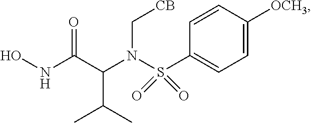

or a pharmaceutically acceptable salt thereof.

In some cases for Formula (I), R¹ is heteroaryl having 5-6 total ring atoms and 1, 2, or 3 heteroatoms selected from N, O, and S, and R² is carboranyl. In some embodiments for Formula (IA), R¹ is heteroaryl having 5-6 total ring atoms and 1, 2, or 3 heteroatoms selected from N, O, and S, and R² is C₁₋₃alkylene-carboranyl. In some cases, R² is CH₂-carboranyl. In various cases, R² is CH₂CH₂-carboranyl. In some embodiments, R² is CH₂CH₂CH₂-carboranyl. Suitable heteroaryl groups for R¹ include, for example, pyridyl, pyrazinyl, pyrimidinyl, pyrrolyl, pyrazolyl, imidazolyl, thiazolyl, triazolyl, oxazolyl, isooxazolyl, thiadiazolyl, oxadiazolyl, furanyl, and thiofuranyl. In some cases, heteroaryl is pyridyl. In various embodiments for Formula (I), R¹ is pyridyl, n is 1, and R² is carboranyl In some embodiments for Formula (IA), R¹ is pyridyl and R² is CH₂-carboranyl. In various embodiments the compound of Formula (I)or (IA) has a structure:

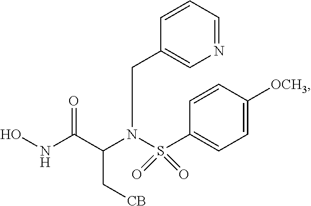

or a pharmaceutically acceptable salt thereof.

In some cases for Formula (I), R¹ is heteroaryl having 5-6 total ring atoms and 1, 2, or 3 heteroatoms selected from N, O, and S, and R² heteroaryl having 5-6 total ring atoms and 1, 2, or 3 heteroatoms selected from N, O, and S, wherein the heteroaryl is substituted with C₁₋₃alkylene-carboranyl. In various cases for Formula (IA), R¹ is heteroaryl having 5-6 total ring atoms and 1, 2, or 3 heteroatoms selected from N, O, and S, and R² is C₁₋₃alkylene-heteroaryl having 5-6 total ring atoms and 1, 2, or 3 heteroatoms selected from N, O, and S, wherein the heteroaryl is substituted with C₁₋₃alkylene-carboranyl. Suitable heteroaryl groups for R¹ include, for example, pyridyl, pyrazinyl, pyrimidinyl, pyrrolyl, pyrazolyl, imidazolyl, thiazolyl, triazolyl, oxazolyl, isooxazolyl, thiadiazolyl, oxadiazolyl, furanyl, and thiofuranyl. In some cases, R¹ is pyridyl. In some cases, R² is Cialkylene-heteroaryl. In various cases, R² is C₂alkylene-heteroaryl. In some embodiments, R² is C₃alkylene-heteroaryl. Suitable heteroaryl groups for R² include, for example, pyridyl, pyrazinyl, pyrimidinyl, pyrrolyl, pyrazolyl, imidazolyl, thiazolyl, triazolyl, oxazolyl, isooxazolyl, thiadiazolyl, oxadiazolyl, furanyl, and thiofuranyl. In some cases, the heteroaryl for R² is triazolyl. In some cases, the heteroaryl for R² is substituted with C₁alkylene-carboranyl. In some embodiments, the heteroaryl for R² is substituted with C₂alkylene-carboranyl. In various embodiments, the heteroaryl for R² is substituted with C₃alkylene-carboranyl. In some embodiments, R² is CH₂-triazolyl substituted with C₃alkylene-carboranyl.

In some cases, the compound of Formula (I) is selected from:

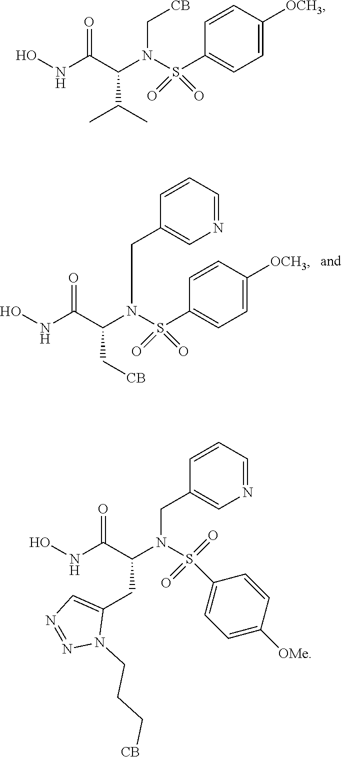

In various embodiments, CB is nido-carboranyl.

In another aspect, disclosed herein is a compound of Formula (II), or a pharmaceutically acceptable salt thereof:

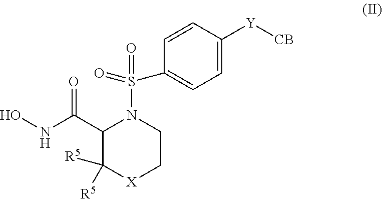

wherein CB is carboranyl; each of X and Y independently is O or S; and each R⁵ independently is H or C₁₋₆alkyl.

In some embodiments, the compound of Formula (II) has a structure of Formula (II′):

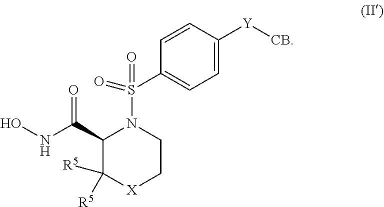

The carboranyl can be any carboranyl as previously described herein. In some cases, the carboranyl is ortho-carboranyl. In various cases, the carboranyl is meta-carboranyl. In some embodiments, the carboranyl is para-carboranyl. In some cases, the carboranyl is nido-carboranyl.

In some embodiments, X is O. In various embodiments, X is S. In some cases, Y is O. In various cases, Y is S. In some embodiments, X is S and Y is S.

In some embodiments each R⁵ is H or C₁₋₃alkyl. In various embodiments each R⁵ is selected from H, methyl, ethyl, propyl and isopropyl. In some embodiments, each R⁵ is H. In various embodiments, each R⁵ is C₁₋₆alkyl. In some embodiments, each R⁵ is C₁₋₃alkyl. For example, each R⁵ can be selected from methyl, ethyl, propyl, and isopropyl. In some cases, each R⁵ is methyl. In various cases, one R⁵ is H and one R⁵ is methyl.

In some cases, each of X and Y is S and each R⁵ is CH₃. In some cases, the compound of Formula (II) is

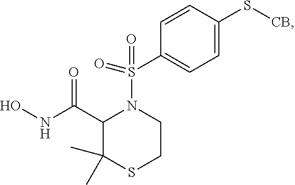

or a pharmaceutically acceptable salt thereof. In various cases, the compound of Formula (II) is selected from:

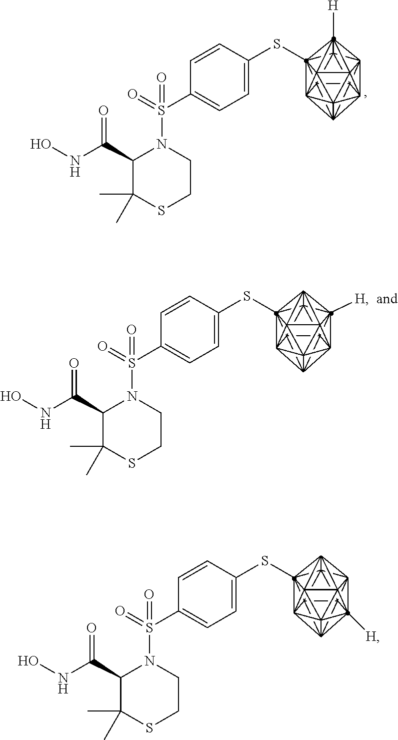

or a pharmaceutically acceptable salt thereof. In various embodiments, CB is nido-carboranyl.

Any of the compounds described herein may be prepared with ¹⁰B-enriched boron to further enhance the efficiency and efficacy of BNCT.

The following selections are envisioned for any of the formulas disclosed herein (e.g., I, I′, IA, IA′, II, II′). In some embodiments, the MMP agents described herein include one or more fluorine (¹⁹F) atoms. The ¹⁹F atom allows the agent to be detected in tumors using magnetic resonance spectroscopy, which demonstrates that the agent has localized to the desired target tissue. Klomp et. al., Magnetic Resonance Med. 50(2):303-8 (2003).

In some cases, the compounds described herein (e.g., the compounds of Formulae I, I′, IA, IA′, II, and II′, or pharmaceutically acceptable salts thereof) act at MMP with an IC₅₀ of about 1000 nM or less. In some embodiments, the compounds described herein (e.g., the compounds of Formulae I, I′, IA, IA′, II, and II′, or pharmaceutically acceptable salts thereof) have an IC₅₀ value for MMP of less than about 100 nM, or less than about 10 nM, or less than about 1 nM. In various cases, the IC₅₀ value of the compounds described herein (e.g., the compounds of Formulae I, I′, II, and II′, or pharmaceutically acceptable salts thereof) is about 1 nM to about 100 nM, or about 0.01 nM to about 1 μM. For example, shown in Table A, carborane-containing BNCT MMP inhibitor, compound 4, exhibits micromolar potency for MMP-1, MMP-2, and MMP-9, demonstrating broad-spectrum potency for the collagenase MMP-1 as well as for the gelatinases MMP-2 and MMP-9 which are known to be upregulated in tumors. It has been demonstrated that compound 4 binds to MMP enzymes, enabling it to deliver and concentrate its concentrated payload of boron atoms in is appended carborane cluster.

### Preparation of the Carborane Hydroxamic Acid MMP Accents

Compounds of the present disclosure can be prepared by any method known to one skilled in the art. In embodiments wherein a piperidine is an intermediate, isolation of the compounds can be accomplished through crystallization of a salt, such as a hydrochloride salt.

Compounds of Formula I and Formula IA can be synthesized in four steps, as shown in Scheme 1, below. The first step involves the reaction of an optionally-protected desired amino acid (e.g., valine) with a desired benzenesulfonyl chloride (e.g, 4-methoxybenzenesulfonyl chloride) to provide an intermediate (e.g., 4-methoxyphenyl)sulfonyl)-D-valine (CAS 68030-19-3)), as described in PCT publication no. WO 1998/003166, which is incorporated by reference in its entirety. The intermediate can be alkylated with a desired functionalized carborane (e.g., bromomethyl carborane), either in a single step or in multiple steps. Coupling the carboxylic acid with O-(tetrahydro-2H-pyran-2-yl)hydroxylamine (“THPONH₂”) followed by acidic removal of the THP protecting group yields the desired carborane-bearing hydroxamates MMP agent.

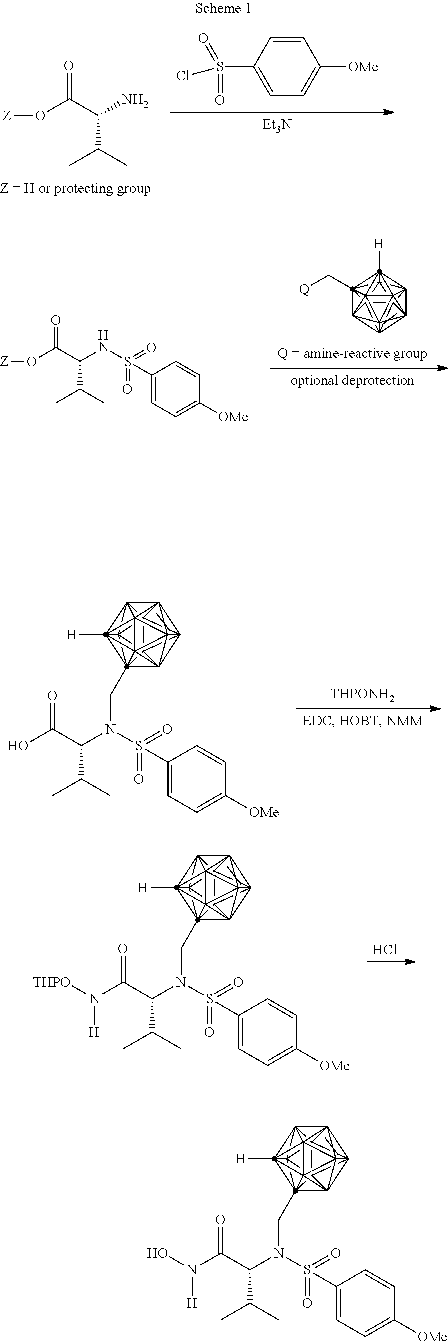

In some cases, the synthesis can be as described in Scheme 1A below, where the intermediate can be alkylated with a desired bromo-substituted carborane (e.g., bromomethyl carborane).

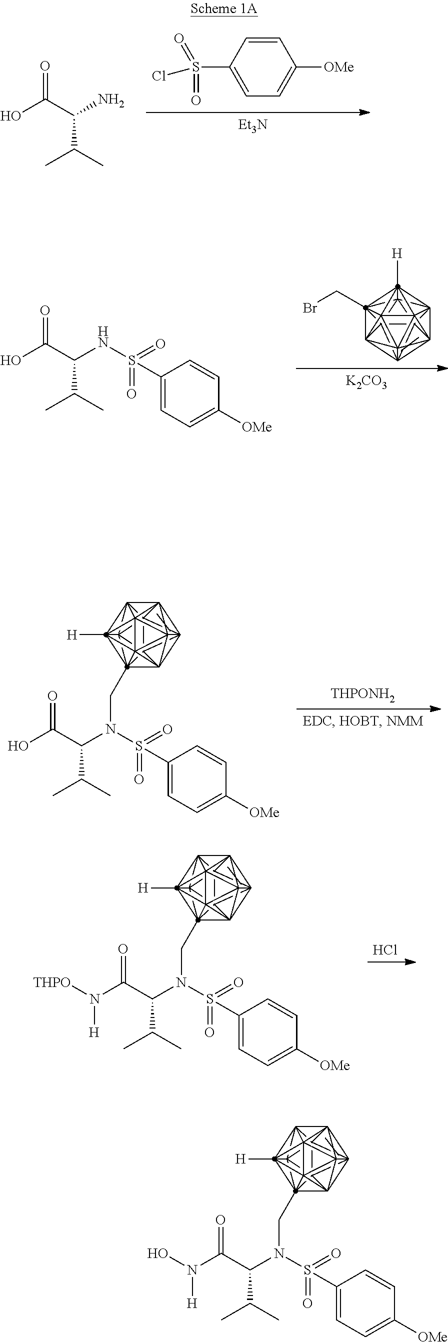

Compounds of Formula I and IA also can be synthesized as shown in Scheme 2, below. In step 1, propargyl glycine is reacted with a desired benzenesulfonyl chloride (e.g., 4-methoxybenzenesulfonyl chloride) to form a sulfonamide (e.g., (R)-2-((4-methyoxyphenyl)sulfonamido)pent-4-ynoic acid, CAS 885104-34-7), which is alkylated to provide a picolyl derivative. The carboxylic acid of the derivative is coupled to THPONH₂, and the resulting intermediate is reacteda borane reagent, optionally having a linker moiety. The THP protecting group can be removed under acidic conditions to afford the desired carborane agent.

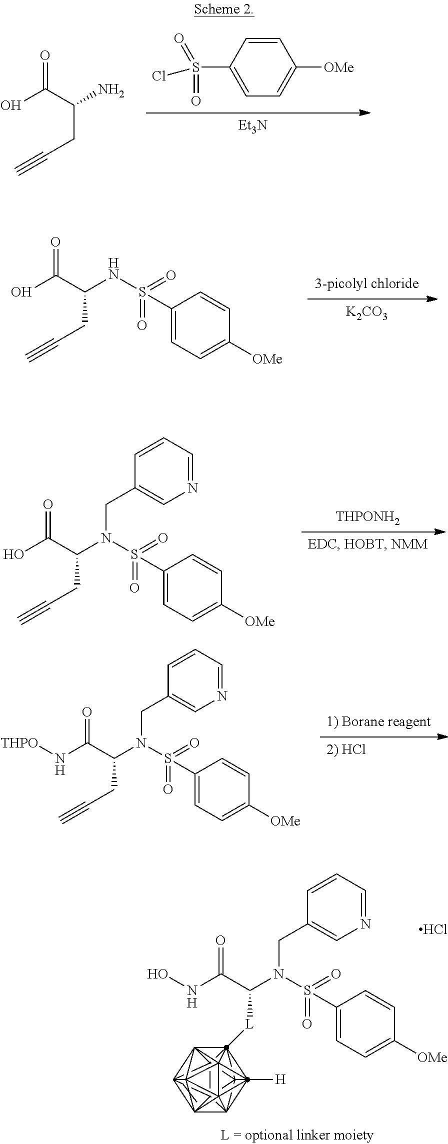

In some cases, the synthesis can be as described in Scheme 2A below, where the intermediate is reacted with the activated decaborane complex, B₁₀H₁₂(MeCN)₂. The reagent B₁₀H₁₂(MeCN)₂ can be prepared in toluene and acetonitrile (typically in excess) and the generated B₁₀H₁₂(MeCN)₂ complex can be isolated as a solid with a moderate shelf-life when stored under an inert atmosphere. Alternatively, the B₁₀H₁₂(MeCN)₂ complex can be prepared in situ for direct carborane synthesis. The THP protecting group can be removed under acidic conditions to afford the desired carborane agent.

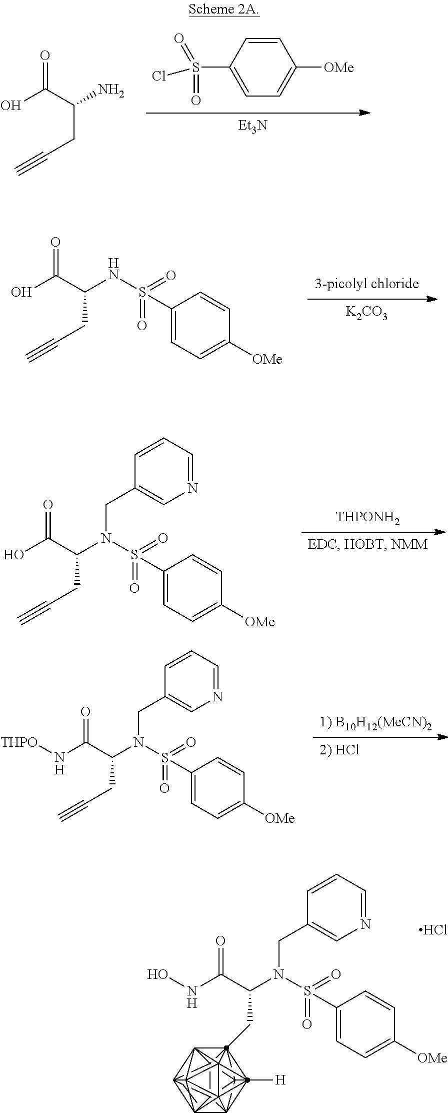

In some cases, the synthesis can be as described in Scheme 2B below, where the intermediate is reacted with an azide-bearing decaborane complex, B₁₀H₁₂(CH₂)₃—N₃. The reaction can be catalyzed, e.g., by a ruthenium complex such as Cp*Ru[COD]Cl, and form a triazole via a 1,3-dipolar azide-alkyne cycloaddition.

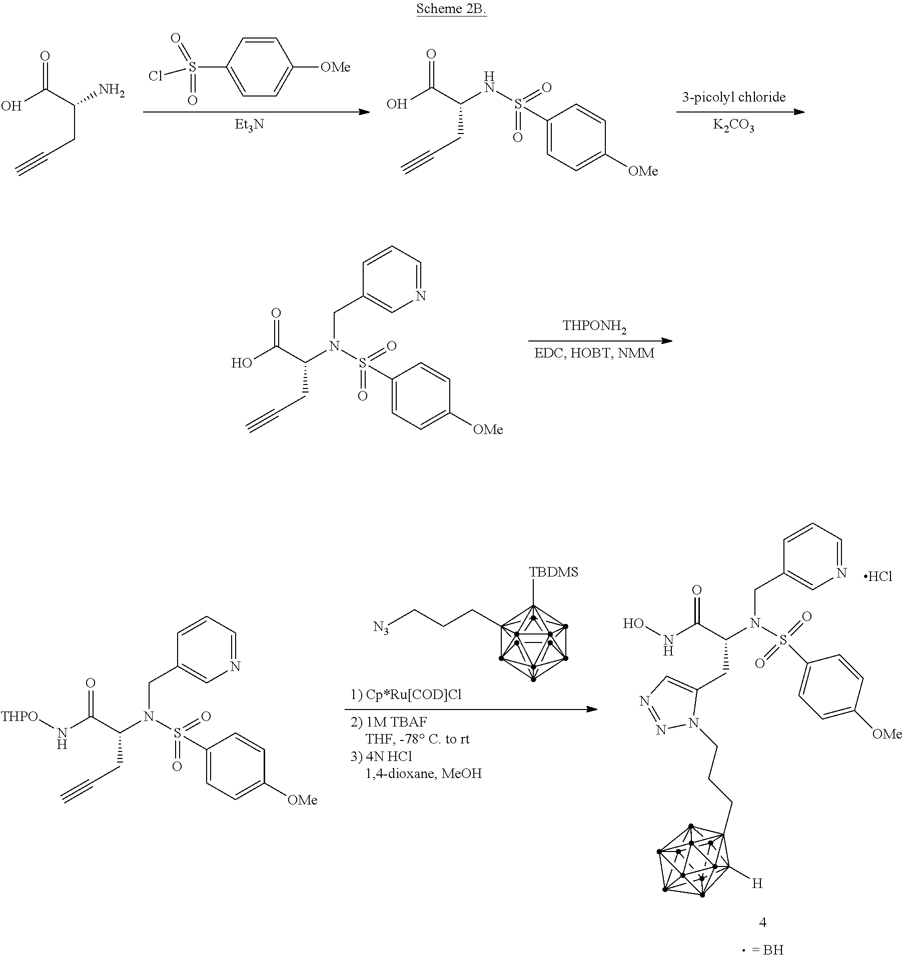

Compounds of Formula II can be synthesized as shown in Scheme 3, below. In the first step, a desired morpholine/thiomorpholine intermediate (e.g., (R)-2,2-dimethylthiomorpholine-3-carboxylic acid, CAS No. 774243-35-5) is prepared, as described in, for example, U.S. Pat. Nos. 5,753,653 and 6,153,757, each of which is incorporated herein by reference. The intermediate is then coupled with THP-protected hydroxylamine to yield the a THP-protected hydroxamate. The hydroxamate can undergo a substitution reaction with a desired sulfonyl chloride (e.g., 4-fluorobenzenesulfonyl chloride) (see, e.g., U.S. Pat. Appl. Publ. No. 2003/0073718, which is incorporated herein by reference), which is then reacted with a carborane-thiol compound to form the desired product after removal of the THP protecting group under acidic conditions.

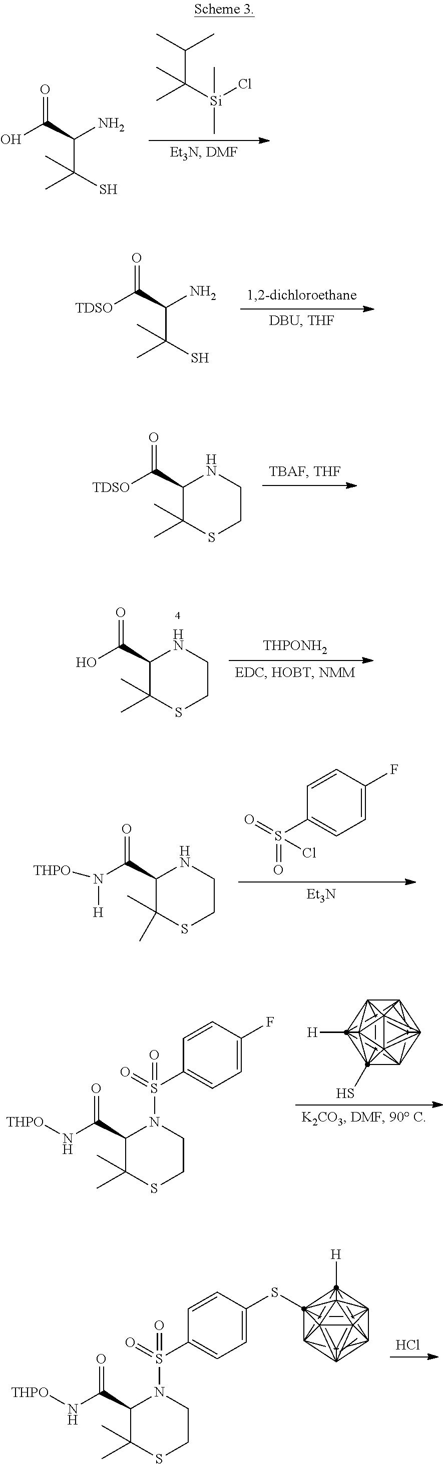

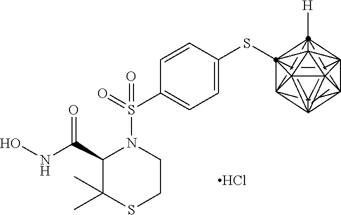

Additional guidance for preparing the compounds described herein can be found in the Examples section.

### Methods of Use

The compounds described herein (e.g., the compounds of Formulae I, I′, IA, IA′, and II or pharmaceutically acceptable salts thereof) can tightly bind to MMP, such as MMP-13, MMP-2, MMP-9, or combinations thereof. Overexpression of MMP has been implicated in a variety of conditions, including tumor growth and metastasis, and in the degradation of articular cartilage in arthritis. Martel-Pelletier et. al Best Practice & Research Clinical Rheumatology 15(5):805-829 (2001). Thus, the compounds described herein are capable of selectively transporting a high concentration of ¹⁰B atoms in the boron-dense carborane to MMPs. Without intending to be bound by any particular theory, when these cells are exposed to an epithermal neutron beam, the ¹⁰B nuclei adsorbs a neutron to form an excited ¹¹B nucleus, which undergoes decay via fission to emit an α-particle (⁴He²⁺) as well as a ⁷Li³⁺ ion, both with high kinetic energy. These highly charged particles can damage the surrounding tissue.

As such, provided herein is a method of delivering ¹⁰B atoms to matrix metalloproteinase (“MMP”) in a cell, comprising contacting the cell with the compound described herein (e.g., a compound of Formulae I, I′, IA, IA′, or II or a pharmaceutically acceptable salt thereof), wherein the compound binds to MMP with an IC₅₀ of 1 μM or less. Yet another aspect of the disclosure relates to a method of inhibiting MMP in a cell comprising contacting the cell with a compound described herein (e.g., a compound of Formula I, Formula II, or pharmaceutically acceptable salts of the foregoing) in an amount effective to inhibit the MMP. In some embodiments, the MMP is MMP-13, MMP-2, MMP-9, or a combination thereof.

The contacting of the cell can occur in vitro or in vivo. In some cases, contacting of the cell occurs in vitro. In other cases, contacting of the cell occurs in vivo. The compounds described herein can contact a cell in vivo by administering a compound described herein to a subject in need of MMP inhibition, such as MMP-13, MMP-2, and/or MMP-9 inhibition. Therefore, the disclosure includes administering one or more of a compound described herein to a subject, such as a human, in need thereof. In some embodiments, the subject suffers from cancer, rheumatoid arthritis, or both.

Further provided are methods of treating or preventing disease in a subject comprising administering to the subject a therapeutically effective amount of a compound described herein (e.g., a compound of Formulae I or II or a pharmaceutically acceptable salt thereof). In some cases, the disease is selected from cancer and rheumatoid arthritis.

In view of the above, in various aspects, the disclosure includes a method of treating a disease in a subject. The method comprises administering a therapeutically effective amount of a compound described herein to a subject in need of MMP inhibition, such that MMP is inhibited. Conditions resulting from overexpression of MMP can include those related to, for example, cancer and rheumatoid arthritis. Use of a compound described herein to treat a condition resulting from overexpression of MMP in a subject, as well as use of a compound described herein in the preparation of a medicament for treating the condition, also are contemplated.

### Pharmaceutical Formulations

Also provided herein are pharmaceutical formulations that include a compound described herein (e.g., a compound of Formula I, I′, IA, IA′, II, II′, or a pharmaceutically acceptable salt thereof), as previously described herein, and one or more pharmaceutically acceptable excipients.

The compounds described herein can be administered to a subject in a therapeutically effective amount. The compounds can be administered alone or as part of a pharmaceutically acceptable composition or formulation. In addition, the compounds can be administered all at once, multiple times, or delivered substantially uniformly over a period of time. It is also noted that the dose of the compound can be varied over time.

The compounds disclosed herein can be administered in combination with one or more additional pharmaceutically active compounds/agents. The additional pharmaceutically active compounds/agents may be small molecules or can be macromolecules such as proteins, antibodies, peptibodies, DNA, RNA or fragments of such macromolecules.

The compounds disclosed herein and other pharmaceutically active compounds, if desired, can be administered to a patient or subject by any suitable route, e.g. orally, rectally, parenterally, (for example, intravenously, intramuscularly, or subcutaneously) intracisternally, intravaginally, intraperitoneally, intravesically, or as a buccal, inhalation, or nasal spray. The administration can be to provide a systemic effect (e.g. enteral or parenteral). All methods that can be used by those skilled in the art to administer a pharmaceutically active agent are contemplated.

Compositions suitable for parenteral injection may comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispersing agents. Microorganism contamination can be prevented by adding various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents delaying absorption, for example, aluminum monostearate and gelatin.

Solid dosage forms for oral administration include capsules, tablets, powders, and granules. In such solid dosage forms, the active compound is admixed with at least one inert customary excipient (or carrier) such as sodium citrate or dicalcium phosphate or (a) fillers or extenders, as for example, starches, lactose, sucrose, mannitol, and silicic acid; (b) binders, as for example, carboxymethylcellulose, alginates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (a) solution retarders, as for example, paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, and tablets, the dosage forms may also comprise buffering agents. Solid compositions of a similar type may also be used as fillers in soft and hard filled gelatin capsules using such excipients as lactose or milk sugar, as well as high molecular weight polyethylene glycols, and the like.

Solid dosage forms such as tablets, dragees, capsules, pills, and granules can be prepared with coatings and shells, such as enteric coatings and others well known in the art. The solid dosage forms may also contain opacifying agents. Further, the solid dosage forms may be embedding compositions, such that they release the active compound or compounds in a certain part of the intestinal tract in a delayed manner. Examples of embedding compositions that can be used are polymeric substances and waxes. The active compound can also be in micro-encapsulated form, optionally with one or more excipients.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, and sesame seed oil, glycerol, tetrahydrofurfuryl alcohol, polyethylene glycols and fatty acid esters of sorbitan, or mixtures of these substances, and the like.

Besides such inert diluents, the composition can also include adjuvants, such as wetting agents, emulsifying and suspending agents, sweetening, flavoring, and perfuming agents. Suspensions, in addition to the active compound, may contain suspending agents, as for example, ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Compositions for rectal administration are preferably suppositories, which can be prepared by mixing the compounds of the disclosure with suitable non-irritating excipients or carriers such as cocoa butter, polyethylene glycol or a suppository wax, which are solid at ordinary room temperature, but liquid at body temperature, and therefore, melt in the rectum or vaginal cavity and release the active component.

The compounds described herein can be administered to a patient or subject at dosage levels in the range of about 0.1 to about 3,000 mg per day. For a normal adult human having a body weight of about 70 kg, a dosage in the range of about 0.01 to about 100 mg per kilogram body weight is typically sufficient. The specific dosage and dosage range that will be used can potentially depend on a number of factors, including the requirements of the patient or subject, the severity of the condition or disease being treated, and the pharmacological activity of the compound being administered. The determination of dosage ranges and optimal dosages for a particular patient or subject is within the ordinary skill in the art.

When a patient or subject is to receive or is receiving multiple pharmaceutically active compounds, the compounds can be administered simultaneously, or sequentially. For example, in the case of tablets, the active compounds may be found in one tablet or in separate tablets, which can be administered at once or sequentially in any order. In addition, it should be recognized that the compositions might be different forms. For example, one or more compound may be delivered via a tablet, while another is administered via injection or orally as a syrup. All combinations, delivery methods and administration sequences are contemplated.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

### EXAMPLES

The following examples are provided for illustration and are not intended to limit the scope of the invention.

### Materials and Methods

All solvents were distilled prior to use and all reagents were used without further purification unless otherwise noted. All synthetic reactions were conducted under an atmosphere of nitrogen. Silica gel 60A, 40-75 μm (200×400 mesh), was used for column chromatography. Aluminum-backed silica gel 200 μm plates were used for TLC. ¹H NMR spectra were obtained using a 500 MHz spectrometer with tetramethylsilane (TMS) as the internal standard. ¹³C NMR spectra were obtained using a 75 or 125 MHz spectrometer. The purity of all compounds was determined to be ≥95% unless otherwise noted by high performance liquid chromatography (HPLC) employing a mobile phase A=5% acetonitrile B in water and a mobile phase B=0.1% TFA in acetonitrile with a gradient of 60% B increasing to 95% over 10 min, holding at 95% B for 5 min, then returning to 60% B and holding for 5 min. HRMS spectra were measured on a TOF instrument by electrospray ionization (ESI). HRMS spectra were collected using a Waters Acquity I class UPLC and Xevo G2-XS QT of mass spectrometer with Waters Acquity BEH C18 column (1.7 μm, 2.1×50 mm). Mobile phase A was 0.05% formic acid in water and mobile phase B was 0.05% formic acid in acetonitrile, and a gradient of 5 to 90% B in Mobile phase A over 5 min was applied.

For the preparation of N-substituted p-methoxyfulfonamide agents, D-valine-t-butyl ester is sulfonylated with p-methoxybenzene sulfonyl chloride in the presence of trimethylamine to give the known sulfonamide [CAS 161315-62-4] which is then alkylated with CB-CH₂Br in the presence of base such as potassium carbonate to provide the N-alkyl derivative. Deptotection with acid followed by coupling of the carboxylic acid with THP-protected hydroxylamine in the presence of EDC provides the THP-protected hydroxamate, which is deprotected with acid to afford the MMP agent for BNCT.

### Example 1a: Preparation of Nitrogen-Substituted p-Methoxysulfonamide BNCT Agents

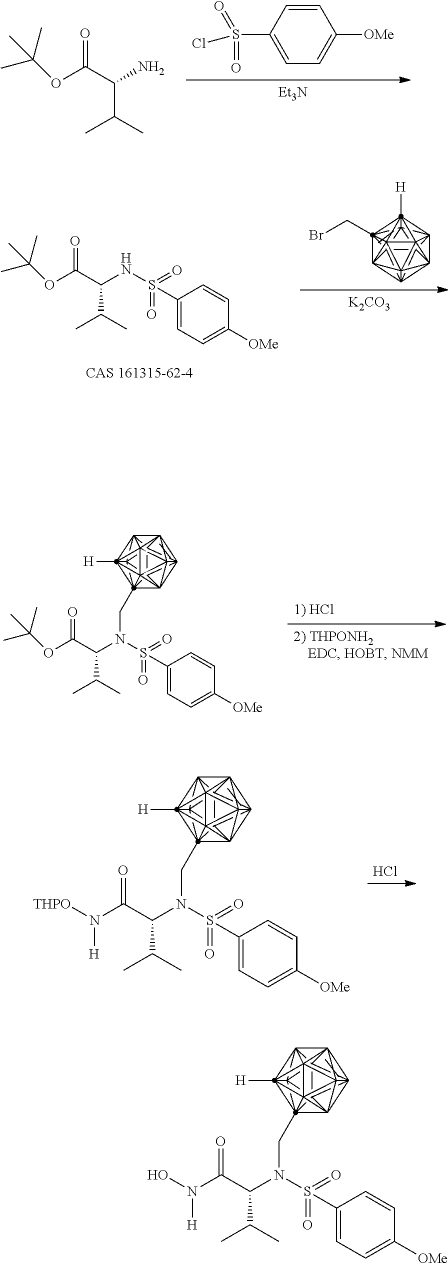

To prepare side chain-substituted p-methoxysulfonamide BNCT agents, D-propargyl glycine is reacted with p-methoxybenzene sulfonyl chloride to provide the known sulfonamide CAS 885104-34-7. Reaction with 3-picolyl chloride in the presence of base such as potassium carbonate yields the N-alkyl derivative.

### Example 1b: Preparation of Nitrogen-Substituted p-Methoxysulfonamide BNCT Agents

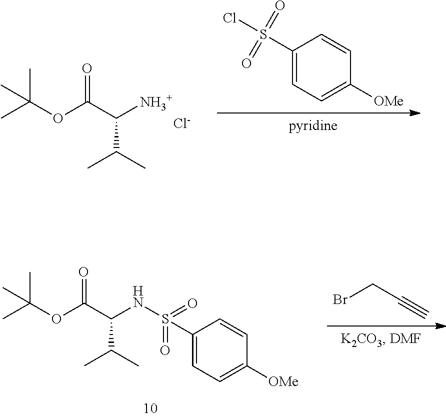

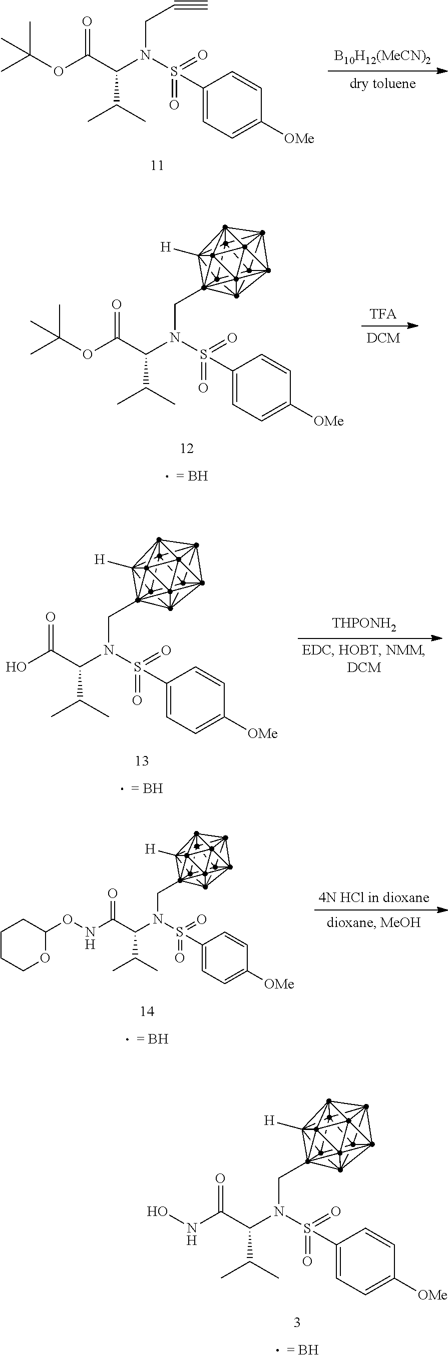

### Synthesis of (R)-tert-Butyl 2-(4-methoxyphenylsulfonamido)-3-methylbutanoate (10)

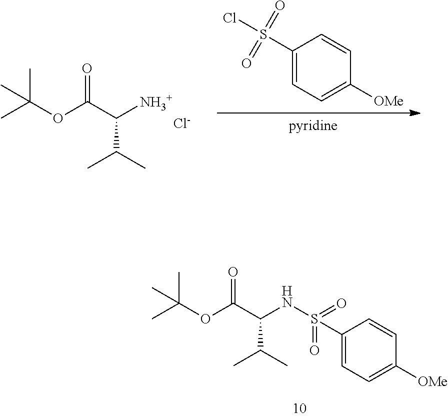

Sulfonamide 10 was prepared according to the literature, and spectral data match reported values. See MacPherson et al., J. Med. Chem. 1997, 40, 2525-2532.

### Synthesis of (R)-tert-Butyl 2-(4-Methoxyphenylsulfonamido-N-(prop-2-yn-1-yl))-3-methylbutanoate (11)

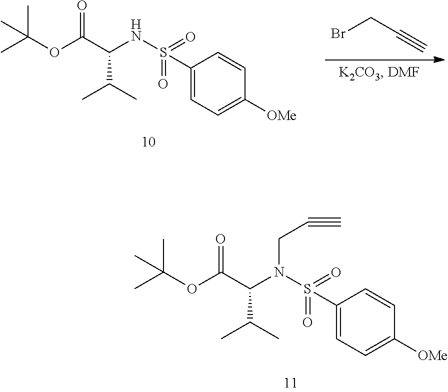

Alkyne 11 was prepared according to the literature, and spectral data match reported values. See Hugenberg et al., J. Med. Chem. 2012, 55, 4714-4727.

### Synthesis of Closo-carborane Complex from (R)-tert-Butyl 2-(4-Methoxyphenylsulfonamido-N-(prop-2-yn-1-yl))-3-methylbutanoate (12)

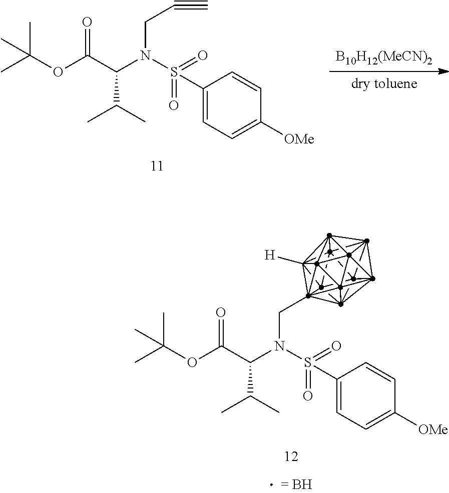

To a solution of enriched decaborane, B₁₀H₁₄, (3.60 g, 31.5 mmol) in anhydrous acetonitrile (103 mL) was added anhydrous toluene (271 mL) and the reaction was warmed to reflux for 1 hour in a pressurized flask under N₂. After cooling, alkyne 11 (7.50 g, 19.6 mmol) was added, and the mixture was warmed to 100° C. and stirred for 24 hours under N₂. The reaction progress was monitored by HPLC for consumption of starting material. Once the reaction was complete, the mixture was cooled and filtered on paper using vacuum filtration. The filter was washed with additional amounts of anhydrous toluene, and the solvent was evaporated under reduced pressure. The crude mixture was then purified by column chromatography (ethyl acetate: hexane=2:8) yielding tent-butyl protected 12.

HPLC analysis: 93.4% AUC. ¹H NMR (500 MHz, CDCl₃) δ7.73 (d, 2H), 7.26 (s, 1H), 6.98 (d, 2H), 4.69 (d, J=17.4 Hz, 1H), 4.64 (s, 1H), 4.10 (d, J=17.5 Hz, 1H), 3.87 (s, 3H), 3.57 (d, J=9.5, 1.7 Hz, 1H), 2.57-1.97 (m, 10H), 1.95 (s, 1H), 1.22 (s, 9H), 1.10 (s, 3H), 0.91 (d, 3H). ¹³C NMR (126 MHz, CDCl₃) δ169.56, 163.82, 130.64, 114.53, 82.68, 75.65, 66.82, 60.38, 55.75, 51.29, 27.75, 19.21. HRMS (ESI-ToF): m/z calcd for C₁₉H₃₇B₁₀NNaO₅S⁺[M+Na]⁺: 514.3597, found 514.3590.

### Synthesis of Carboxylic Acid (13)

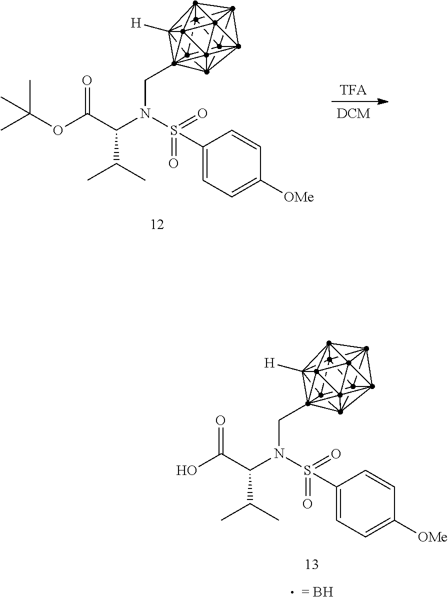

To a solution of tert-butyl protected 12 (1.66 g, 3.33 mmol) in anhydrous methylene chloride (28 mL) was added trifluoroacetic acid (11 mL) and the mixture was stirred for 2 hours at room temperature. The reaction progress was monitored by HPLC for consumption of starting material. Once the reaction was complete, the solvent was evaporated under reduced pressure, and the traces of trifluoroacetic acid were removed by adding toluene (7.0 mL) and concentrating again under reduced pressure to yield carboxylic acid 13.

HPLC analysis: 93.5% AUC. ¹H NMR (500 MHz, DMSO-d⁶) δ12.90 (s, 1H), 7.81 (d, J=8.5 Hz, 2H), 7.10 (d, J=8.4 Hz, 2H), 5.03 (s, 1H), 4.54 (d, J=17.4 Hz, 1H), 4.15 (d, J=17.5 Hz, 1H), 3.85 (s, 3H), 3.59 (d, J=9.6 Hz, 1H), 2.72-1.68 (m, 10H), 1.82 (s, 1H), 0.99 (s, 3H), 0.85 (d, 3H). ¹³C NMR (126 MHz, DMSO-d⁶) δ171.06, 163.25, 130.46, 128.43, 114.39, 76.33, 66.21, 62.16, 55.69, 50.98, 20.97, 18.96. HRMS (ESI-ToF): m/z calcd for C₁₅H₃₀B₁₀NO₅S⁺[M+H]⁺: 434.3022, found 434.2983.

### Synthesis of THP-Protected Hydroxamate (14)

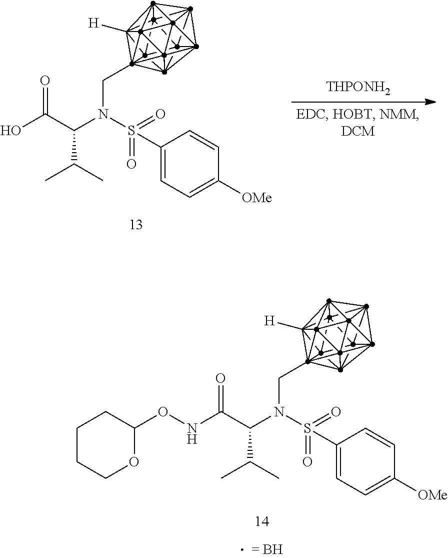

To a solution of carboxylic acid 13 (1.40 g, 3.16 mmol) in anhydrous methylene chloride (80 mL) were added 1-hydroxybenzotriazole hydrate (HOBT, 468 mg, 3.46 mmol), 4-methylmorpholine (NMM, 2.0 mL) O-tert-butylhydroxylamine hydrochloride (1.22 g, 9.71 mmol), and N-(3-dimethylaminopropyl)-N′-ethylcarbodiimide hydrochloride (EDC, 862 mg, 4.50 mmol). The mixture was vigorously stirred overnight at room temperature under N₂. The reaction progress was monitored by HPLC for consumption of starting material. Once the reaction was complete, the reaction mixture was diluted with DI water, and extracted with dichloromethane (3×). The combined organic layers were washed with brine, and dried over Na₂SO₄. The solvent was evaporated under reduced pressure, and the crude was further purified by column chromatography (ethyl acetate: hexane=1:1) yielding THP-hydroxamate 14.

HPLC analysis: 92.1% AUC. HRMS (ESI-ToF): m/z calcd for C₂₀H₃₉B₁₀N₂O₆S⁺[M+H]⁺: 533.3772, found 533.3662.

### Synthesis of (3)

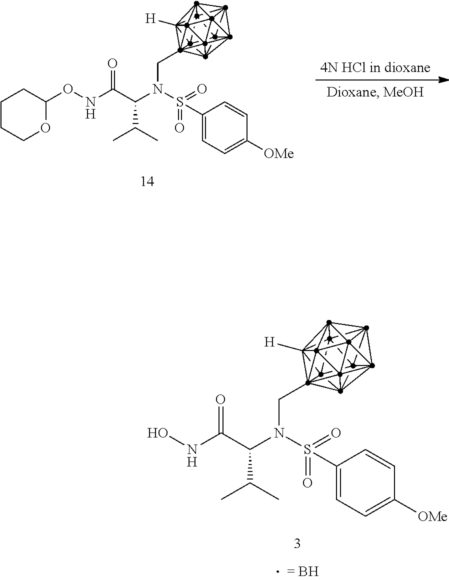

To a solution of THP-protected 14 (1.15 g, 2.11 mmol) were added anhydrous dioxane (12 mL), anhydrous methanol (8.0 mL), and 4N HCl in dioxane (0.40 mL) and the reaction was stirred at room temperature for 2.5 hours. The reaction progress was monitored by HPLC for consumption of starting material. Once the reaction was complete, the reaction mixture was concentrated under reduced pressure to give a crude oil. The crude product was dissolved in a minimal amount of dichloromethane (1.0 mL) and methanol (2.0 mL), then slowly pipetted into stirring solution of hexane (80 mL) and diethyl ether (20 mL). The product precipitated out of the solution and was vacuum filtered through a fritted glass filter to obtain hydroxamate 3.

HPLC analysis: 95.3% AUC. ¹H NMR (500 MHz, DMSO-d⁶) δ10.43 (s, 1H), 8.79 (s, 1H), 7.86 (d, J=8.8 Hz, 2H), 7.09 (d, J=8.5 Hz, 2H), 5.30 (d, J=17.7 Hz, 1H), 5.01 (s, 1H), 3.85 (s, 3H), 3.46 (d, J=9.8 Hz, 1H), 2.38-1.53 (m, 10H), 1.78 (s, 1H), 1.02 (s, 3H), 0.79 (s, 2H), 0.68 (s, 1H). HRMS (ESI-ToF): m/z calcd for C₁₅H₃₀B₁₀N₂NaO₅S⁺[M+Na]⁺: 473.3097, found 473.3107.

### Example 2: Preparation of Side Chain-Substituted p-Methoxysulfonamide BNCT Agents

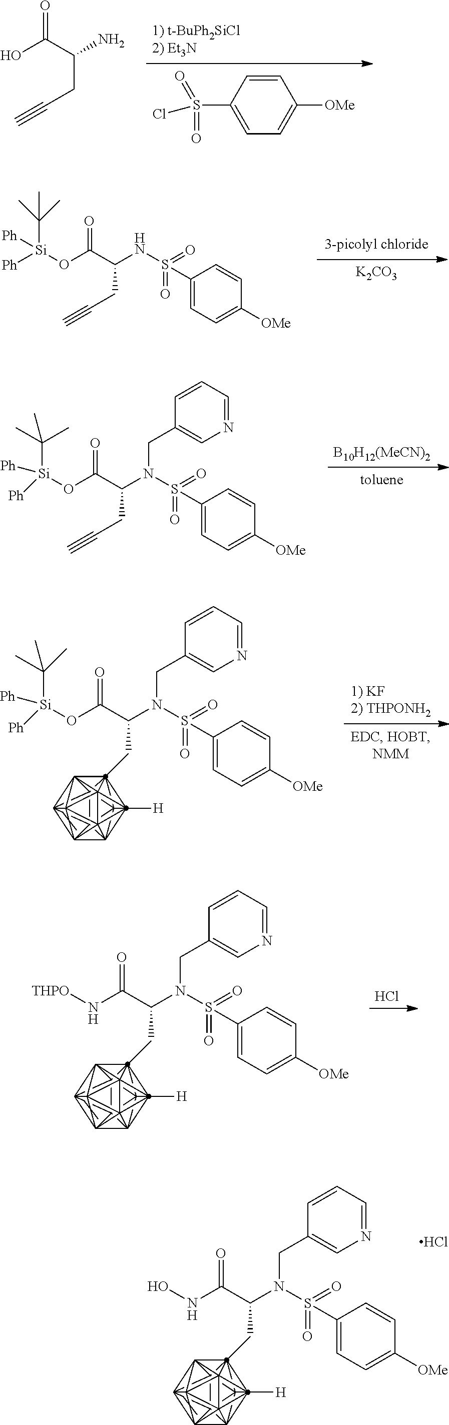

For preparation of the side chain substituted p-methoxybenzenesulfonamide BNCT agents, D-propargylglycine is protected as the t-butyldiphenylsilyl ester and then reacted with p-methoxybenzenesulfonyl chloride to afford the corresponding sulfonamide. Alkylation with 3-picolyl chloride and potassium carbonated yields the N-alkylated sulfonamide. The propargyl group is then reacted with decaborane to give the CB-derivative. Deprotection of the silyl ester with potassium fluoride followed by coupling with THPONH2 in the presence of EDC as coupling agent gives the THP-protected penultimate derivative, which is deprotected with acid to give the requisite BNCT agent as the HCl salt.

### Example 3: Preparation of Thiomorpholine BNCT Accents

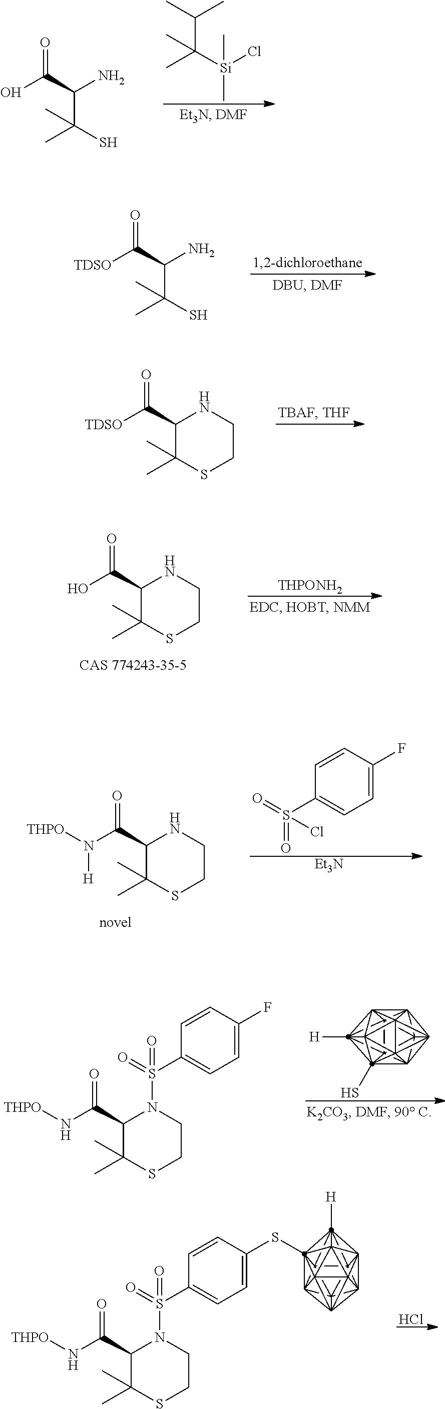

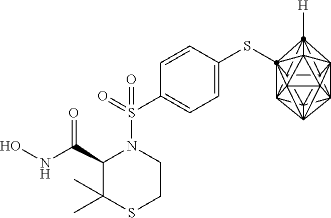

For preparation of the thiomorpholine BNCT agents, D-mercaptovaline is protected as the t-butyldiphenylsilyl ester, then bis-alkylated with 1,2-dichloroethane and DBU in DMF. Removal of the silyl protecting group with TBAF is followed by coupling with THPONH2 in the presence of EDC to give the THP-protected ester. Reaction with p-fluorobenzenesulfonyl chloride affords the sulfonamide, which undergoes a SNAR reaction with the mercapto-CB derivative in the presence of base to give the penultimate derivative which is deprotected with acid to give the requisite thiomorpholine BNCT agent.

### Example 4: Preparation of Side Chain-Substituted p-Methoxysulfonamide BNCT Agents

### Synthesis of Compound 21

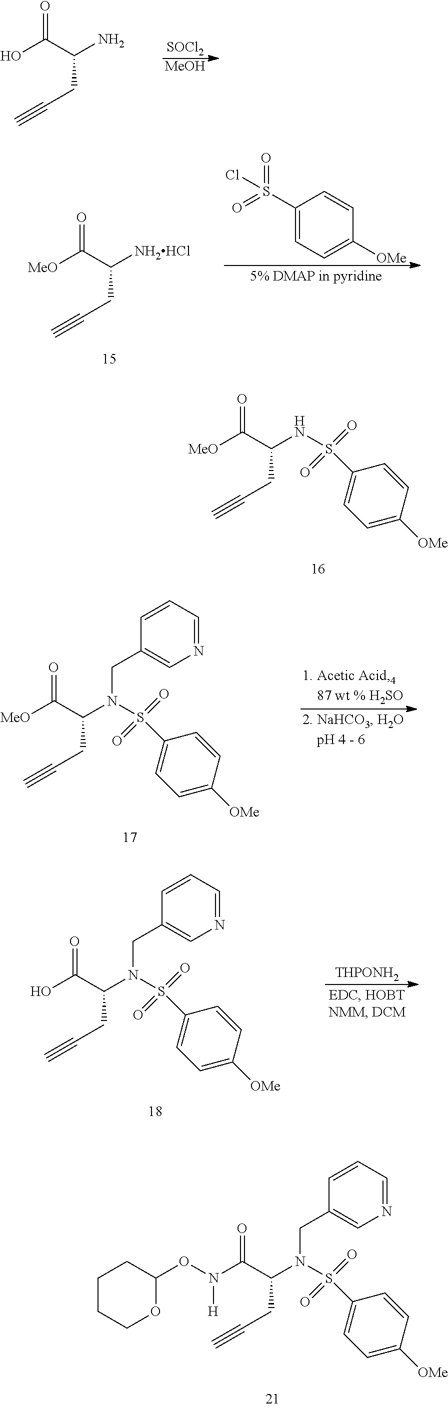

### Synthesis of Methyl (R)-2-aminopent-4-ynoate hydrochloride (15)

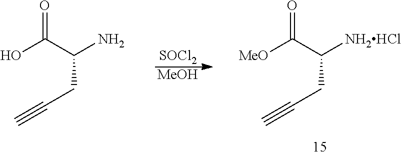

Amine hydrochloride 15 was prepared according to the literature, and spectral data match reported values. See Ourailidou et al., Bioorg. Med. Chem. 2017, 25, 847-856.

### Synthesis of Methyl (R)-2-((4-methoxyphenyl) sulfonamido) pent-4-ynoate (16)

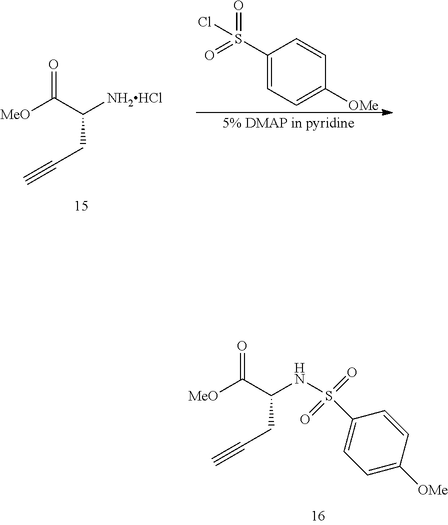

To a solution of amine hydrochloride 15 (1.37 g, 8.37 mmol) in anhydrous pyridine (3.4 mL) and dimethylaminopyridine (148 mg, 1.23 mmol) at 0° C. was slowly added 4-methoxybenzenesulfonyl chloride (1.90 grams, 9.21 mmol). The solution was allowed to warm to room temperature and stirred for one day under N₂. The reaction progress was monitored by TLC (ethyl acetate: hexane=1:1) for consumption of 4-methoxybenzenesulfonyl chloride. Once the reaction was complete, the mixture was diluted with methylene chloride, washed 2N HCl (10×) and DI water (5×). The organic layer was then washed with brine (2×) and dried with Na₂SO₄. The solvent was evaporated under reduced pressure to provide sulfonamide 16.

HPLC analysis: 98.1% AUC. ¹H NMR (500 MHz, CDCl₃) δ7.72 (d, 2H), 6.90 (d, 2H), 5.33 (d, J=8.8 Hz, 1H), 4.03 (dt, J=9.5, 4.9 Hz, 1H), 3.80 (s, 3H), 3.56 (s, 3H), 2.61 (qdt, J=17.0, 4.9, 2.0 Hz, 2H), 1.96 (s, 1H) ¹³C NMR (126 MHz, CDCl₃) δ169.09, 162.11, 130.32, 128.36, 113.23, 76.46, 71.28, 54.63, 52.93, 51.92, 23.06.

### Synthesis of Methyl (R)-2-((4-methoxy-N-(pyridine-3-ylmethyl) phenyl) sulfonamido) pent-4-ynoate (17)

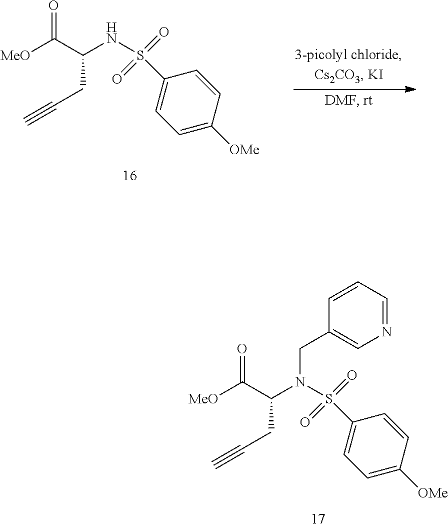 [SMILES: C#CC[C@@H](NS(=O)(=O)c1ccc(OC)cc1)C(=O)OC ; C#CC[C@H](C(=O)OC)N(Cc1cccnc1)S(=O)(=O)c1ccc(OC)cc1]

To a solution of sulfonamide 16 (300 mg, 1.01 mmol) in anhydrous DMF (6.9 mL) was added Cs₂CO₃ (700 mg, 2.15 mmol), followed by potassium iodide (200 mg, 1.2 mmol) and 3-picolyl chloride (252 mg, 1.54 mmol, recrystallized from ethyl alcohol). The solution was stirred for one day under N₂ at room temperature. The reaction progress was monitored by HPLC for generation of desired product, and the reaction was stopped early to avoid formation of undesired impurities. The mixture was then diluted with DI H₂O and extracted with EA (3×). The organic layers were combined and washed with 5% NaHCO₃ (2×), followed by DI H₂O (2×), brine (2×) and dried with Na₂SO₄. The solvent was evaporated under reduced pressure, and the crude was further purified by column chromatography (ethyl acetate: dichloromethane: hexane=1:1.16:1.16) to provide N-picolylsulfonamide 17.

HPLC analysis: 97.8% AUC. ¹H NMR (500 MHz, CDCl₃) δ8.50 (d, 2H), 7.82-7.73 (m, 3H), 7.23 (dd, J=7.9, 4.8 Hz, 1H), 6.95 (d, 2H), 4.70 (dd, J=8.6, 6.1 Hz, 1H), 4.57 (d, J=16.1 Hz, 1H), 4.46 (d, J=16.2 Hz, 1H), 3.87 (s, 3H), 3.56 (s, 3H), 2.76 (ddd, J=7.2, 6.2, 2.7 Hz, 1H), 2.61 (ddd, J=17.2, 8.6, 2.7 Hz, 1H), 1.96 (s, 1H). ¹³C NMR (126 MHz, CDCl₃) δ169.54, 163.17, 149.66, 149.21, 136.46, 132.17, 131.32, 129.83, 123.3, 114.07, 71.80, 58.73, 55.66, 52.50, 47.46, 21.08.

### Synthesis of Compound 18

Compound 17 was dissolved in acetic acid and allowed to stir at room temperature until complete dissolution was achieved. The solution of ester was diluted with 87% sulfuric acid, stirred for 10 minutes at room temperature, then glacial acetic acid was added and the solution was left to stir for two hours. The collected crude product was concentrated at 60° C. under reduced pressure to remove acetic acid. The resulting sulfuric acid residue was diluted with methylene chloride and then placed in a cooling bath (10° C.). The mixture was carefully diluted with saturated sodium bicarbonate solution followed by slow addition of solid sodium bicarbonate to achieve a pH of 3.5. The mixture was further diluted with USP purified water and then extracted with DCM. The combined organic layers were dried with sodium sulfate, filtered, and concentrated to give compound 18.

### Synthesis of Compound 21

To a solution of carboxylic acid 18 in anhydrous methylene chloride were added 1-hydroxybenzotriazole hydrate, 4-methylmorpholine O-tert-butylhydroxylamine hydrochloride and N-(3-dimethylaminopropyl)-N′-ethylcarbodiimide hydrochloride. The mixture was vigorously stirred overnight at room temperature under N₂. The reaction progress was monitored by HPLC for consumption of starting material. Once the reaction was complete, the reaction mixture was diluted with DI water, and extracted with dichloromethane (3×). The combined organic layers were washed with brine, and dried over Na₂SO₄. The sample was further purified by column chromatography to provide the desired compound 21.

### Preparation of Compound 25

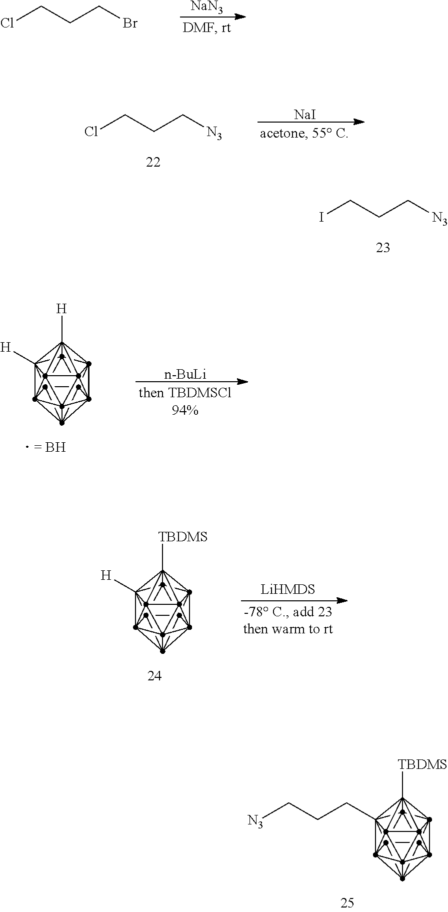

### Synthesis of 1-azido-3-chloropropane (Compound 22)

Compounds 22 and 23 were synthesized according to a general procedure reported in Choi et al,. Angewandte Chemie 2017, 129, 7528-7532. To a 250 mL round bottomed flask was added 100 mL of anhydrous DMF and 10.02 g of 1-bromo-3-chloropropane and then 4.2 g of sodium azide. The reaction was placed in an ambient temperature water bath and stirred overnight (16 h) at room temperature. The reaction mixture was diluted with 50 mL of Et₂O and 50 mL USP purified water, stirred 2-3 minutes then separated the organic layer (top). Extracted the bottom aqueous layer with Et₂O (2×60 mL). The combined organic layers were washed with USP purified water (3×50 mL), dried with sodium sulfate, filtered and concentrated at 25-30° C. under reduced pressure to give a colorless oil (7.21 g, 95% mass balance) which was taken forward without any further purification to give a mixture of 1-azido-3-chloropropane as the major product that contained a small amount of 1-azido-3-bromopropane, and which was carried on as such into the next step.

¹H NMR (CDCl₃, 400 MHz): δ3.64 (t, 2H, J=8.0 Hz), 3.51 (t, 3H, J=8.0 Hz), 2.02 (q, 2H, J=12.0, 4.0 Hz)

### Synthesis of 1-azido-3-iodopropane (Compound 23)

Compounds 22 and 23 were synthesized according to a general procedure reported in Choi et al,. Angewandte Chemie 2017, 129, 7528-7532. To a 1000 mL 3-necked round bottomed flask was added 19.35 g of sodium iodide, 7.5 g of crude 1-azido-3-chloropropane and 190 mL of acetone. The vessel was purged with dry nitrogen, covered with aluminum foil, and heated to 52° C. After 40 h, the reaction was allowed reaction to cool to room temperature. The reaction mixture as a yellow slurry was filtered over a pad of Celite, and the funnel and flask were with acetone (˜100 mL), then concentrated the yellow filtrate on a rotovap at 25-30° C. to remove acetone. After concentration, an orange-yellow residue was obtained (oily solids, 26.2 g). Hexane (50 mL) was added to the oil/solid residue (yellowish-orange) which changed the color to a greenish solid. The slurry was stirred overnight at room temperature then passed over a short silica plug (65 g) packed in n-hexane, then the plug was eluted with n-hexane to collect fractions (each 50-70 mL). Appropriate fractions were combined and concentrated at 25° C. to give iodo azide 23.

¹H NMR (CDCl₃, 400 MHz): δ3.44 (t, 2H), 3.25 (t, 2H), 2.04 (quintet, 2H).

### Synthesis of TBDMS Carborane (Compound 24)

Compound 24 was synthesized according to a general procedure reported in Ahrens et al., J. Med. Chem. 2011, 54, 2368-2377. 3.00 g of o-carborane, 12 mL anhydrous toluene, and 6 mL anhydrous Et₂O were combined. Stirred at room temperature until completely dissolved then cooled to <5° C. Added n-BuLi solution (1.66 M, 13.2 mL) over about 5 minutes. Removed cooling bath after 5 minutes and allowed to store at room temperature. After 2.5 hours, solid TBDMSCI (3.47 g) was added at room temperature as one portion which addition was endothermic. After 22.5 h, the reaction slurry was analyzed by TLC (80% hexane and 20% Et₂O) and showed a trace amount of starting material and the reaction was deemed complete. The reaction mixture was quenched with 30 mL of USP purified H₂O, then extracted with Et₂O (3×30 mL). The combined organic layers were dried with MgSO₄, filtered and concentrated to give crude product 7.07 g as a pale yellow oil. The crude oil was purified over silica gel (140 g, 60-200 micron) eluting with n-hexane and 10% Et₂O/hexane to give the silyl-protected carborane 24.

mp 64-66° C. ¹H NMR (CDCl₃, 400 MHz): δ3.44 (bs,1H), 2.87-1.54 (m, 10H), 1.02 (s, 9H), 0.23 (s, 6H). ¹¹B NMR (Decoupled, 100 MHz): δ=0.34, −1.76, −7.02, −10.73, −12.31, −13.26. ¹¹B NMR (Coupled, 100 MHz): δ=1.01, −0.94, −2.57, −6.29, −7.87, −9.99, −11.62, −12.41, −13.2, −14.26.

### Synthesis of TBDMS Propyl Azido Carborane (Compound 25)

Compound 25 was synthesized according to a general procedure reported in Choi et al,. Angewandte Chemie 2017, 129, 7528-7532. To a dry 100 mL round bottomed flask under a nitrogen atmosphere was added anhydrous THF (18 mL) and 1M LiHMDS (9.7 mL). The mixture was cooled to −78° C. A solution of TBDMS carborane (2.00 g) in anhydrous THF (10 mL) was added to the cryogenic mixture via syringe over 5 min such that the temperature was maintained ≤−65° C. The reaction mixture was allowed to stir an additional 5 minutes at −78° C. then allowed to warm to 0° C., stirred an additional 1.25 h at 0° C., and cooled to −78° C. A solution of 1-azido-3-iodopropane (2.15 g) in anhydrous THF (12 mL) was added over 3 minutes at −78° C. The reaction was allowed reaction to stir at −78° C. for 10 minutes then allowed to warm to room temperature and stirred an additional 1.25 hours at ambient temperature. The reaction was cooled to 0° C., quenched with USP purified water (5 mL), concentrated under reduced pressure, extracted with diethyl ether (2×20 mL). The combined organic layers were dried with sodium sulfate, filtered and concentrated under reduce pressure to give a crude yellow oil (3.13 g). The crude oil (3.13 g) was dissolved in DCM/n-hexane (3.5 mL, 25/75, v/v) and passed through a large silica plug (40 g) packed in DCM/n-hexane (25/75, v/v). The silica plug was flushed with DCM/n-hexane (200 mL, 25/75, v/v) to collect 8 fractions (each about 10-15 mL). Fractions 2-6 were combined and concentrated to give carborane azide 25.

mp 41-43° C. ¹H NMR (CDCl₃, 400 MHz): δ3.32 (t, 2H), 3.15-1.5 (m, 14H), 1.07 (s, 9H), 0.34 (s, 6H). ¹³C NMR (CDCl₃, 20 MHz): δ80.6, 76.5, 50.9, 35.3, 29.7, 27.7, 20.5, −2.3. ¹¹B NMR (Decoupled, 100 MHz): δ=0.29, −3.76, −7.29, −10.18. ¹¹B NMR (Coupled, 100 MHz): δ=0.99, −0.56, −3.13, −4.62, −6.57, −8.16, −9.48, −11.24.

### Preparation of Compound 4

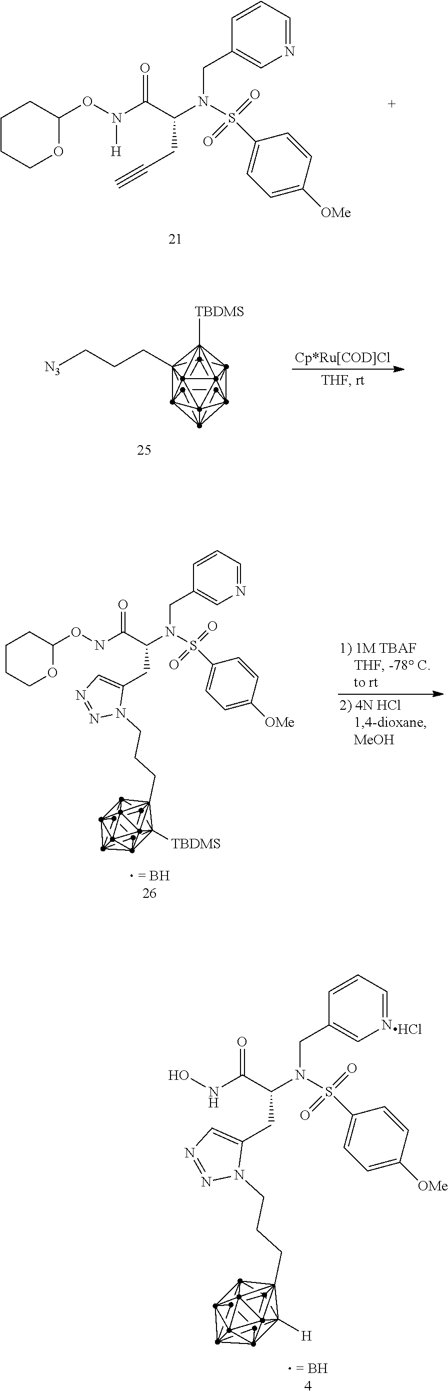

### Synthesis of Compound 4

To a 20 mL round bottomed flask was added alkyne 21 (1.29 mmol), carboranyl azide 25 (1.35 mmol), Cp*RuCl(cod) (65 mg, 0.17 mmol), magnetic stir bar, and THF (13 mL). The mixture was sparged with nitrogen for 5 minutes, then allowed to stir at room temperature for 24 hours which TLC and HPLC analysis showed complete consumption of alkyne starting material. The reaction mixture was concentrated under reduced pressure to give crude product. The crude product was dissolved in ethyl acetate (6 mL) and loaded onto three separate preparative thin layer chromatography plates (20×20 cm) and eluting with 100% ethyl acetate to give purified 1,5-triazole 26.

To TBDMS triazole 26 (139 mmol) was added anhydrous THF (2 mL) and the resulting mixture was cooled solution to −78° C. To the cryogenic mixture was added a solution of 1M TBAF in THF (0.17 mL) over approximately 30 seconds. After 5 minutes, the cooling bath was removed and then the reaction was permitted to warm to room temperature. The reaction mixture was concentrated to a residue which was dissolved in ethyl acetate (2 mL) and washed with water (1 mL, pH 7-7.5). The aqueous phase was extracted ethyl acetate (1 mL). The combined organic layers were washed with water (pH 7-7.5, 1 mL), dried with sodium sulfate, filtered, and concentrated under reduced pressure to give crude product. The crude product is dissolved in ethyl acetate (1 mL) and passed through a silica plug eluting with ethyl acetate and the plug was flushed with ethyl acetate to afford desired THP-protected triazole. To this product in a 10 mL vial under nitrogen atmosphere is added anhydrous 1,4-dioxane (0.9 mL)/methanol (0.1 mL) and the mixture is allowed to stir until complete dissolution was achieved. To the solution was added 4N HCl in 1,4-dioxane (0.14 mL) and the reaction is allowed to stir for 2 h at room temperature. The reaction mixture is concentrated under reduced pressure at 30° C. to give a crude product. The crude product is dissolved in dichloromethane (1 mL) and diethyl ether (3 mL) was added to generate a white slurry. The slurry was allowed to stir at ambient temperature for 1.5 hours, filtered, and the filter cake was washed with diethyl ether (2 mL) and n-heptane (5 mL), pulled dry under nitrogen, and further dried in vacuo at room temperature to provide the title 1-triazole carborane 4.

### Example 5: Assay for Inhibition of MMP

Serial dilutions of a compound were prepared with 10% DMSO and 5 μL of the dilution was added to a 50 μL reaction so that the final concentration of DMSO is 1% in all of reactions.

The enzymes were diluted in 50 mM HEPES buffer pH 7.4, 10 mM CaCl₂, 0.05% Brij-35, and 1 mM APMA for activation at 37° C. for 2 hours. The enzymatic reactions were conducted in duplicate at room temperature for 30 minutes in a 50 μL mixture containing 50 mM HEPES buffer, pH7.4, 10 mM CaCl₂, 0.05% Brij-35, an MMP substrate (390 MMP FRET Substrate I (Mca-PLGL-Dpa-AR-NH2 from AnaSpec)), an MMP enzyme (MMP-1, MMP-2, or MMP-9), and BNCT MMP inhibitor (compound 4).

Fluorescence intensity was measured at an excitation of 328 nm and an emission of 393 nm using a Tecan Infinite M1000 microplate reader.

Phosphatase activity assays were performed in duplicate at each concentration. The fluorescent intensity data were analyzed using the computer software, Graphpad Prism. In the absence of the compound, the fluorescent intensity (Fₜ) in each data set was defined as 100% activity. In the absence of enzyme, the fluorescent intensity (F_{b}) in each data set was defined as 0% activity. The percent activity in the presence of each compound was calculated according to the following equation: %activity=(F−F_{b})/(Fₜ−F_{b}), where F=the fluorescent intensity in the presence of the compound.

The values of % activity versus a series of compound concentrations were then plotted using non-linear regression analysis of Sigmoidal dose-response curve generated with the equation Y=B+(T−B)/1+10^{((Log EC50-X)×Hill Slope)}, where Y=percent activity, B=minimum percent activity, T=maximum percent activity, X=logarithm of compound and Hill Slope=slope factor or Hill coefficient. The IC₅₀ value was determined by the concentration causing a half-maximal percent activity. IC₅₀ values for Compound 4 and an NNGH (N-Isobutyl-N-(4-methoxyphenylsulfonyl)glycyl Hydroxamic Acid) standard are presented in Table A, below:

TABLE A

| | IC₅₀ (μM) or Percentage Inhibition | | |
|---|---|---|---|
| Compound | MMP1 | MMP2 | MMP9 (Q279R) |
| Compound 4 | 3.0 | 2.5 | 1.3 |
| NNGH | 0.13 | 0.004 | 0.004 |

We claim:

1. A compound having a structure of Formula (I), or a pharmaceutically acceptable salt thereof: 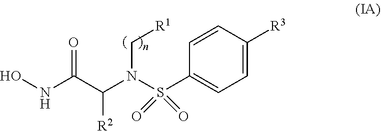
wherein
n is 1, 2, or 3;
R¹ is either (a) heteroaryl having 5-6 total ring atoms and 1, 2, or 3 heteroatoms selected from N, O, and S, or (b) carboranyl;
R² is either (a) C₁₋₆alkyl, (b) C₁₋₃alkylene-carboranyl, or (c) C₁₋₃alkylene-heteroaryl having 5-6 total ring atoms and 1, 2, or 3 heteroatoms selected from N, O, and S, wherein the heteroaryl is substituted with C₁₋₃alkylene-carboranyl;
with the proviso that
(i) when R¹ is (b), then R² is (a), and
(ii) when R² is (b) or (c), then R¹ is (a);
R³ is H, OH, halo, C₁₋₆alkyl, C₁₋₆haloalkyl, C₁₋₆alkoxyalkyl, C₁₋₃alkyleneC₆₋₁₀aryl, OC₁₋₆alkyl, OC₁₋₆haloalkyl, OC₁₋₆alkoxyalkyl, OC₀₋₃alkyleneC₆₋₁₀aryl, or N(R⁴)₂; and
R⁴ is H or C₁₋₃alkyl.

2. The compound or salt of claim 1, having a structure of Formula (IA′):

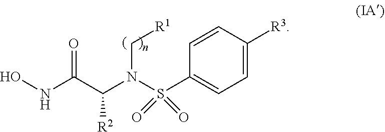

3. The compound or salt of claim 1 or 2, wherein n is 1.

4. The compound or salt of claim 1 or 2, wherein n is 2.

5. The compound or salt of any one of claims 1-4, wherein the carboranyl is ortho-carboranyl.

6. The compound or salt of any one of claims 1-4, wherein the carboranyl is meta-carboranyl.

7. The compound or salt of any one of claims 1-4, wherein the carboranyl is para-carboranyl.

8. The compound or salt of any one of claims 1-7, wherein the carboranyl is nido-carboranyl.

9. The compound or salt of any one of claims 1-8, wherein R³ is C₁₋₆alkyl, C₁₋₆haloalkyl, OC₁₋₆alkyl, or OC₁₋₆haloalkyl

10. The compound or salt of claim 9, wherein R³ is C₁₋₃alkyl, C₁₋₃fluoroalkyl, OC₁₋₃alkyl, or OC₁₋₃haloalkyl.

11. The compound or salt of claim 10, wherein R³ is CH₃, CF₃, OCH₃, or OCF₃.

12. The compound or salt of any one of claims 1-8, wherein R³ is C₁₋₃alkyleneC₆₋₁₀aryl or OC₀₋₃alkyleneC₆₋₁₀aryl.

13. The compound or salt of claim 12, wherein R³ is O-phenyl.

14. The compound or salt of claim 13, wherein the phenyl is unsubstituted.

15. The compound or salt of claim 13, wherein the phenyl is substituted.

16. The compound or salt of claim 1, wherein n is 1 and R³ is OCH₃, OCF₃, or O-phenyl.

17. The compound or salt of any one of claims 1-16, wherein R¹ is carboranyl and R² is C₁₋₆alkyl.

18. The compound or salt of claim 17, wherein R² is C₁₋₃alkyl.

19. The compound or salt of claim 18, wherein R² is isopropyl.

20. The compound or salt of any one of claims 1-16, wherein R¹ is heteroaryl having 5-6 total ring atoms and 1, 2, or 3 heteroatoms selected from N, O, and S, and R² is C₁₋₃alkylene-carboranyl or C₁₋₃alkylene-heteroaryl having 5-6 total ring atoms and 1, 2, or 3 heteroatoms selected from N, O, and S, wherein the heteroaryl is substituted with C₁₋₃alkylene-carboranyl.

21. The compound or salt of claim 24, wherein R¹ is pyridinyl.

22. The compound or salt of claim 20 or 21, wherein R² is C₁₋₃alkylene-carboranyl.

23. The compound or salt of claim 22, wherein R² is CH₂-carboranyl.

24. The compound or salt of claim 20 or 21, wherein R² is C₁₋₃alkylene-heteroaryl having 5-6 total ring atoms and 1, 2, or 3 heteroatoms selected from N, O, and S, wherein the heteroaryl is substituted with C₁₋₃alkylene-carboranyl.

25. The compound of salt of claim 24, wherein heteroaryl is pyridyl, pyrazinyl, pyrimidinyl, pyrrolyl, pyrazolyl, imidazolyl, thiazolyl, triazolyl, oxazolyl, isooxazolyl, thiadiazolyl, oxadiazolyl, furanyl, or thiofuranyl.

26. The compound or salt of claim 25, wherein heteroaryl is triazolyl.

27. The compound or salt of any one of claims 24-26, wherein R² is trizolyl substituted with C₃alkylene-carboranyl.

28. The compound or salt of claim 1 having a structure: 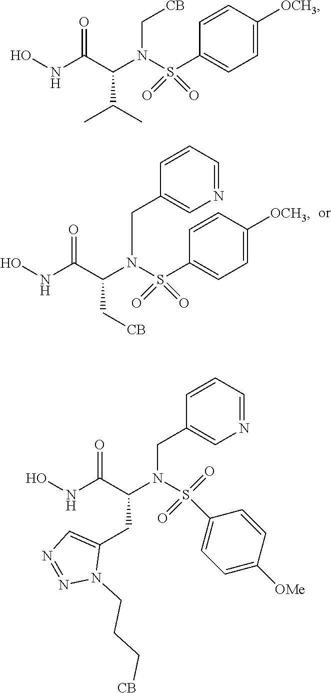
or a pharmaceutically acceptable salt thereof, wherein CB is carboranyl.

29. The compound or salt of claim 28, wherein CB is nido-carboranyl.

30. A compound having a structure of Formula (II), or a pharmaceutically acceptable salt thereof: 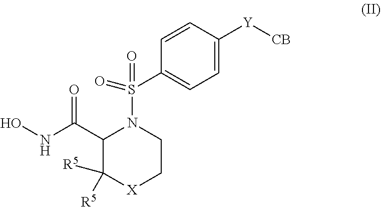
wherein
CB is carboranyl;
each of X and Y independently is O or S; and
each R⁵ independently is H or C₁₋₆alkyl.

31. The compound of claim 30, having a structure of Formula (II′):

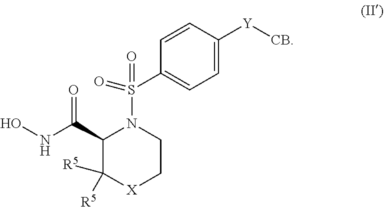

32. The compound or salt of claim 31, wherein the carboranyl is ortho-carboranyl.

33. The compound or salt of claim 31, wherein the carboranyl is meta-carboranyl.

34. The compound or salt of claim 31, wherein the carboranyl is para-carboranyl.

35. The compound or salt of any one of claims 31-34, wherein the carboranyl is nido-carboranyl.

36. The compound or salt of any one of claims 31-35, wherein X is S.

37. The compound or salt of any one of claims 31-36, wherein Y is S.

38. The compound or salt of any one of claims 31-37, wherein each R⁵ independently is H.

39. The compound or salt of any one of claims 31-37, wherein each R⁵ independently is C₁₋₆alkyl.

40. The compound or salt of claim 39, wherein each R⁵ independently is C₁₋₃alkyl.

41. The compound or salt of claim 40, wherein each R⁵ independently is CH₃.

42. The compound or salt of any one of claims 31-37, wherein one R⁵ is H and one R⁵ is CH₃.

43. The compound or salt of claim 31, wherein each of X and Y is S and each R⁵ is CH₃.

44. The compound or salt of claim 31, wherein the compound is 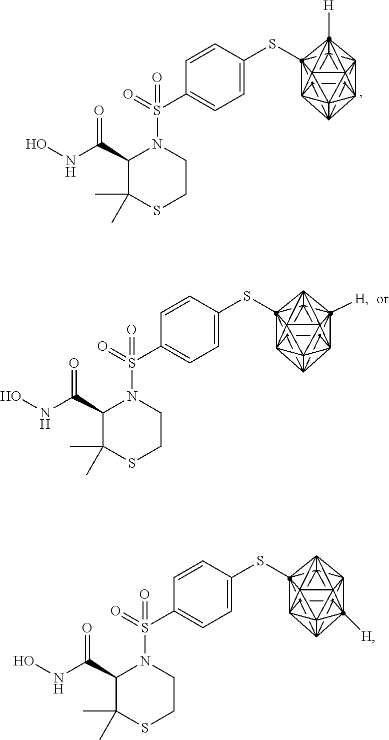
or a pharmaceutically acceptable salt thereof.

45. A pharmaceutical formulation comprising the compound or salt of any one of claims 1 to 44 and a pharmaceutically acceptable excipient.

46. A method of delivering ¹⁰B atoms to matrix metalloproteinase (“MMP”) in a cell, comprising contacting the cell with the compound or salt of any one of claims 1 to 44, wherein the compound binds to MMP with an IC₅₀ of 1 μM or less.

47. The method of claim 46, wherein the MMP is MMP-13, MMP-2, MMP-9, or a combination thereof.

48. The method of claim 46 or 47, wherein the contacting occurs in vivo.

49. The method of any one of claims 46-48, wherein the contacting comprises administering to a subject in need thereof.

50. The method of claim 49, wherein the subject suffers from cancer, rheumatoid arthritis, or both.

51. A method of treating a disease in a subject comprising administering to the subject a therapeutically effective amount of the compound of any one of claims 1-44 or the pharmaceutical formulation of claim 45.

52. The method of claim 51, wherein the disease is cancer or rheumatoid arthritis.
